(19) United States
(12) Patent Application Publication
(10) Pub. No.: US 2011/0281831 A1
(43) Pub. Date: Nov. 17, 2011

(54) NOVEL DERMACEUTICAL CREAM MADE USING SODIUM FUSIDATE, ANTIFUNGALS AND STEROIDS

(75) Inventors: Vanagamudi Subramaniam Sulur, Chennai (IN); Madhavan Srinivasan, Chennai (IN); Neelakandan Narayanan Chulliel, Chennai (IN); Haridas Sankar, Mumbai (IN); Balkrishnana Selvaraj, Chennai (IN)

(73) Assignee: APEX LABORATORIES PRIVATE LIMITED, CHENNAI, TN (IN)

(21) Appl. No.: 13/144,933

(22) PCT Filed: Jan. 20, 2010
(86) PCT No.: PCT/IB10/50243

§ 371 Date: Jul. 18, 2011

(30) Foreign Application Priority Data
Jan. 21, 2009 (IN) 133/MUM/2009

(51) Int. Cl. A61K 31/575; A61P 17/00; A61P 29/00; A61P 31/04; A61P 31/10

(52) U.S. Cl. 514/170

(57) ABSTRACT

The invention discloses a dermaceutical cream containing antifungal agents, steroids and an antibacterial agent in the form of Fusidic acid, which Fusidic acid is formed in situ from Sodium Fusidate as the starting raw material, wherein Sodium Fusidate is converted into Fusidic acid under oxygen-free environment. The cream of the present invention has greater shelf-life stability and the finer particle size of the API than the conventional creams containing Fusidic acid. The cream of the present invention contains Fusidic acid as the API that has been formed in situ from Sodium Fusidate, and steroids in a cream base comprising an acid, a co-solvent, an emulsifier and a waxy material along with water, preferably purified water.

### FIELD OF INVENTION

The present invention relates to primary & secondary bacterial skin infections, fungal skin infections and inflammations and in particular it relates to the single dose treatment using a steroid and antifungal cream that also contains an antibacterial agent in the form of a Fusidic acid wherein the Fusidic acid has been made using Sodium Fusidate as the starting Active Pharmaceutical Ingredient (API).

### BACKGROUND OF THE INVENTION

Use of steroids to alleviate inflammation, irritation and itching caused by skin ailments is well known. It is also well known that use of steroids compromises patient's immune system and exposes them to bacterial and fungal infections. Single dose therapies containing steroids, antifungals and antibacterials are well known.

Numerous single dose treatments, both topical and systemic, are currently employed for the treatment of above skin inflammations. Topical and systemic inflammatory treatment compositions typically employ a combination of corticosteroids in a base component. The active ingredients typically comprise Corticosteroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like.

Fungal infections sometimes follow the use of antibiotics, which kill nonpathogenic as well as pathogenic bacteria, thereby providing a free field in the body for fungal invasion.

Numerous treatments both topical and systemic are currently employed for the treatment of fungal infections. Topical and systemic fungal infections, treatment compositions typically employ antifungal agents as active ingredients in a base component.

The active ingredients typically comprise antifungal agents such as Miconazole Nitrate, Terbinafine Hydrochloride, Ketoconazole and the like.

Numerous treatments, both topical and systemic, are available for the primary and secondary skin infection caused by sensitive Gram +ve organisms such as Staphylococcus aureus, Streptococcus spp etc. Topical and systemic bacterial infection treatment compositions typically employ at least one active pharmaceutical ingredient (API) in combination with a base component. In the cream form, the APIs typically comprise an antibiotic/antibacterial such as Fusidic acid and the like.

In the currently available Fusidic acid creams, Fusidic acid in fine powder form is used as source API. The small particle size enhances its dermal contact by providing a large specific surface area and penetration, and provides a smooth feel on application to skin. However, a serious shortcoming of the fine size of Fusidic acid particles is that it presents an enormous surface area for contact and reaction with molecular Oxygen during manufacture, handling, and processing of the cream. This has serious implications to its chemical stability and results in rapid reduction in potency of the API (Fusidic acid) in the final cream formulation. Degradation due to oxidation is a major cause of instability of currently available Fusidic acid creams. Table 1 show that the degradation in the API samples (Fusidic acid) exposed to oxygen ranged between 7.7% and 11% for conditions ranging from room temperature to 45° C. when analysed at three months of exposure period at the above conditions.

It is known that greater the exposure time of Fusidic acid as the raw API to Oxygen, greater the limitations on stabilising Fusidic acid in a formulation. However, there is no published data on the stability of Fusidic acid over a period of time.

As an alternative to Fusidic acid, Sodium Fusidate is known to have been used to make dermaceutical medicaments for topical application. However, these are in the form of ointment rather than cream. Drawbacks of ointments over creams are well known and it's generally preferable to use creams rather than ointments for topical application.

Several aspects of Fusidic acid as an API are known:
It is thermolabile
It is available in cream formulations
It can be obtained from Sodium Fusidate by dissolving the latter in an aqueous phase and adding acid to the solution, whereby Fusidic acid precipitates. However, the Fusidic acid precipitate is difficult to process into a cream form first due to its coarse and uneven particle size and second retrieving Fusidic acid from wet cake involves drying and further handling which deteriorates the Fusidic acid due to exposure to oxygen
The stability of the API in a Fusidic acid cream is unreliable due to the thermolabile nature of Fusidic acid

Stabilization of medicaments containing Fusidic acid against oxidation involves observing a number of stringent precautionary procedures during manufacture and storage. These include:
replacing Oxygen in pharmaceutical containers with inert gases such as Nitrogen, Carbon dioxide, Helium and the like
avoiding contact of the medicament with heavy metal ions which catalyze oxidation,
storing the API at reduced temperatures throughout its shelf life before processing

In practice this means stricter controls during the manufacture as well as storage of such API (storing it typically at 2° C. to 8° C. in air-tight containers throughout their shelf life). There is therefore a need to provide a Fusidic acid cream in which Fusidic acid will be of greater stability at the time of the manufacture of the cream, and which will sustain its stability at an acceptable level throughout its shelf life.

There's a need to provide dermaceutical cream containing steroids, antifungals and an antibacterial in the form of Fusidic acid, and in which Fusidic acid will be of greater stability at the time of the manufacture of the cream, and which will sustain its stability at an acceptable level throughout its shelf life.

### OBJECTS AND ADVANTAGES OF THE INVENTION

It is therefore one object of the present invention to provide a cream which contains Fusidic acid as the active API but which has greater stability of the API throughout its shelf life.

It is a further objective of the present invention to provide a dermaceutical cream containing at least one steroid, at least one antifungal and an antibacterial agent in the form of Fusidic acid, in which Fusidic acid will be of greater stability at the time of the manufacture of the cream, and which will sustain its stability at an acceptable level throughout its shelf life.

### BRIEF SUMMARY OF THE INVENTION

The invention discloses a dermaceutical cream containing steroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like, antifungals such as Miconazole Nitrate, Terbinafine Hydrochloride, Ketoconazole and the like. and an antibacterial agent in the form of Fusidic acid, which Fusidic acid is formed in situ from Sodium Fusidate as the starting raw material, wherein Sodium Fusidate is converted into Fusidic acid under oxygen-free environment. The cream of the present invention has greater shelf-life stability and the finer particle size of the API than the conventional creams containing Fusidic acid. The cream of the present invention contains Fusidic acid as the API that has been formed in situ from Sodium Fusidate, and steroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like, and antifungals such as Miconazole Nitrate, Terbinafine Hydrochloride, Ketoconazole and the like in a cream base comprising an acid, a co-solvent, an emulsifier and a waxy material along with water, preferably purified water.

### DETAILED DESCRIPTION OF THE INVENTION

We discussed earlier the known aspects of the topical preparations that have Fusidic acid and Sodium Fusidate as the APIs. It is evident from the current state of knowledge that:
Creams containing Fusidic acid that are made using Sodium Fusidate as starting API are not available.
Creams containing Fusidic acid that are made using Sodium Fusidate along with steroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like and antifungals such as Miconazole nitrate, Terbinafine Hydrochloride, Ketoconazole and the like as starting APIs are not available.
There is no published data on the stability of Sodium Fusidate as the API.
Sodium Fusidate is not considered to be inherently more stable as an API than Fusidic acid.

In the face of this, it has been surprisingly discovered that Sodium Fusidate as an API is significantly more stable than Fusidic acid and that Fusidic acid deteriorates more rapidly than Sodium Fusidate.

There is no published data on the stability of Sodium Fusidate as the API. The applicant carried out experiments on Sodium Fusidate to evaluate its stability. It can be seen from Table 2 that the degradation of Sodium Fusidate over a temperature range of room temperature to 45° C. ranged between 2.45% and 6%.

Tables 1 and 2 also show the comparison between the stability of the Fusidic acid and Sodium Fusidate as raw APIs. The study was carried out using an in-house HPLC method developed by the applicant, which the applicant believes is a true stability-indicating method as opposed to the titration method suggested in British Pharmacopoeia (BP). This is because the BP method does not differentiate between the intact API and the degraded form.

### Stability Analysis of Fusidic Acid

TABLE 1
Results Of 3 Months Old Fusidic Acid (API) Analysis By
Stability Indicating HPLC Method And Titration Method

| | | | Fusidic Acid | | Percentage | | |
|---|---|---|---|---|---|---|---|
| | | | Assay | | Drop | | |
| S. | | *Initial | (%) | | (%) | | Re- |
| No | Conditions | (%) | Titration | HPLC | Titration | HPLC | marks |
| 1 | RT (O) | 100.6 | 99.21 | 92.93 | 1.39 | 7.67 | API |
| 2 | RT (C) | | 99.02 | 94.37 | 1.58 | 6.23 | analysed |
| 3 | 45° C. (O) | | 98.52 | 89.52 | 2.08 | 11.08 | After 3 |
| 4 | 45° C. (C) | | 99.10 | 92.12 | 1.50 | 8.48 | Months |

Name of the Sample: FUSIDIC ACID BP
Pack: Open (O) & Closed (C) Petri dish

### Stability Analysis of Sodium Fusidate

TABLE 2
Results Of 3 Months Old Sodium Fusidate (API) Analysis By
Stability Indicating HPLC Method And Titration Method

| | | | Sodium Fusidate | | | | |
|---|---|---|---|---|---|---|---|
| S. | | *Initial | Assay(%) | | Percentage (%) | | Re- |
| No | Conditions | (%) | Titration | HPLC | Titration | HPLC | marks |
| 1 | RT (O) | 98.7 | 97.71 | 96.25 | 0.99 | 2.45 | API |
| 2 | RT (C) | | 98.85 | 97.67 | −0.15 | 1.03 | analysed |
| 3 | 45° C. (O) | | 97.07 | 92.65 | 1.63 | 6.05 | After 3 |
| 4 | 45° C. (C) | | 97.16 | 92.96 | 1.54 | 5.74 | Months |

Name of the Sample: Sodium Fusidate BP
Pack: Open (O) & Closed (C) Petri dish

In both studies the * Initial denotes the results of the samples tested at the time of receipt of the API from the supplier.

It can be observed from Tables 1 and 2 that:
In the case of Fusidic Acid, there is about 7.7% loss in 3 Months at room temperature (open condition) and about 11% loss in 3 Months at 45° C. (open condition).
In the case of Sodium Fusidate, there is about 2.5% loss in 3 Months at room temperature (open condition) and about 6% loss in 3 Months at 45° C. (open condition).

The data thus shows that Sodium Fusidate as an API is more stable than Fusidic acid.

The applicants explored the possibility of making a cream (rather than an ointment) using Sodium Fusidate (rather than Fusidic acid) and steroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like, and antifungals such as Miconazole Nitrate, Terbinafine Hydrochloride, Ketoconazole and the like. Although Sodium Fusidate has been used in dermaceutical applications, it has not been possible to make creams that use Sodium Fusidate. This is because of the inherent alkalinity of Sodium Fusidate (pH 7.5 to 9), which means it cannot be used in a cream form therefore all products manufactured using Sodium Fusidate as starting material are ointments. A dermaceutical cream that uses Sodium Fusidate, antifungals and steroids would exploit the benefit of the fact that Sodium Fusidate is more stable than Fusidic acid and it would also provide a cream formulation which is far superior in its application qualities than an ointment. It would thus fill an existing need for a cream that has better stability than currently available creams containing Fusidic acid, antifungals and steroids.

The applicant therefore surprisingly discovered that in order to achieve greater stability of the API in a dermaceutical cream, Sodium Fusidate rather than Fusidic acid may be used as the starting API during the cream's manufacture. Using Sodium Fusidate as starting material eliminates the drawback associated with the manufacture and storage of existing Fusidic acid creams.

The applicant has also discovered that the Fusidic acid, antifungal and Steroids cream prepared using Sodium Fusidate as the starting APIs showed good chemical stability, and efficacy.

The application discloses a cream containing Steroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like, antifungals such as Miconazole Nitrate, Terbinafine Hydrochloride, Ketoconazole and the like and Fusidic acid (the API) that has been prepared using Sodium Fusidate as the starting API, in which Fusidic acid forms in-situ under totally oxygen free environment by slow addition of an acid, into a molecular dispersion form (due to the presence of a co-solvent) at the intermediate stage, and which Fusidic acid regenerates as an extremely fine dispersion when added to a final cream base, thereby resulting in a finely and homogeneously dispersed Fusidic acid in the final cream. All these operations are performed in an environment free of atmospheric oxygen. The cream of the present invention contains Fusidic acid as the API that has been formed in situ from Sodium Fusidate, steroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like, antifungals such as Miconazole Nitrate, Terbinafine Hydrochloride, Ketoconazole and the like in a cream base comprising an acid, a co-solvent, a preservative, an emulsifier and a waxy material along with water, preferably purified water.

The APIs which may be employed in the present invention as starting APIs are either acid-based actives or their salts well known in the art of treating bacterial primary & secondary infections, fungal infections and inflammations. Examples of suitable acid-based actives or their salts which may be used include, but are not limited to Sodium Fusidate, steroids such as Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like, antifungals such as Miconazole Nitrate, Terbinafine Hydrochloride, Ketoconazole and the like.

These acid-based active compounds or their salts require a base component to be used in the pharmaceutical composition that uses the compounds, since the compounds cannot, by themselves, be deposited directly on to human skin due to their harshness.

The cream base of the present invention optionally further comprises an ingredient selected from a group comprising a buffering agent, an anti oxidant, a chelating agent, and a humectant, or any combination thereof.

The present invention provides a novel cream that has been produced using Sodium Fusidate as the starting raw material, and which cream contains Fusidic acid of high therapeutic efficacy and of chemical stability that is generally superior to the commercially available creams containing Fusidic acid.

The Fusidic acid, antifungal and steroids cream of the present invention has been manufactured in a totally oxygen free environment under purging with inert gas and applying vacuum. Under these conditions, the Sodium Fusidate is converted in situ into Fusidic acid. The cream of the present invention is used in the treatment of bacterial skin infections, fungal infections and inflammations.

The pH of the product of the present invention is from about 3 to 6. On the other hand, Sodium Fusidate ointments that are commercially available are greasy and cosmetically non elegant.

It is essential that the active drug penetrates the skin for the optimum bio-dermal efficacy. The particle size of the active drug plays an important role here. It is necessary that the active drug is available in a finely dispersed form for the product to be being efficacious. Also this is to be achieved in the safe pH compatible environment of skin (4.0 to 6.0). To achieve all these, it is essential to choose proper vehicles or co-solvents for the dissolution or dispersion of the drug.

The product of the present invention is efficacious due to the pronounced anti-inflammatory, antifungal, antibacterial activity of the steroids, antifungals and regenerated Fusidic acid which is available in reduced particle size than the conventional products, and in a finely dispersed form.

The inventor has screened different co-solvents such as Propylene Glycol, Hexylene Glycol, PolyEthyleneGlycol-400 & the like and dissolved the Sodium Fusidate in one of above co-solvents varying from about 5% (w/w) to 40% (w/w) under inert gas purging and under vacuum and converted to Fusidic acid in-situ by adding an acid such as HCl, H₂SO₄, HNO₃, Lactic acid and the like from about 0.005% (w/w) to about 0.5% (w/w) under stirrng and obtained Fusidic acid in more stabilized and solution form, which makes our final product in a cream base which easily penetrates the skin and highly efficacious, and also highly derma compatible by having a pH of about 3.0 to about 6.0.

The stability of the product is confirmed by the stability studies performed for 3/6 months as per ICH guidelines.

### Experimental Data

APIs-stability experiments were carried out (see tables 3-74) using the product of the present invention. Tests were carried out to observe (or measure as appropriate) the physical appearance of the product, the pH value and assay of the APIs over a period of time. Each gram of product of the present invention used for the tests contained Sodium Fusidate in the amount required to produce 2% (w/w) Fusidic acid in the finished product and appropriate amount of steroids and antifungals as mentioned below.

### Steroids

i. Betamethasone Valerate—0.12% (w/w)
ii. Fluticasone Propionate—0.05% (w/w)
iii. Mometasone Furoate—0.1% (w/w)
iv. Dexamethasone Acetate—0.1% (w/w)
v. Hydrocortisone Acetate—1.0% (w/w)\
vi. Clobetasol Propionate—0.05% (w/w)
vii. Beclomethasone Dipropionate—0.025% (w/w)
viii. Betamethasone Dipropionate—0.05% (w/w)

### Antifungals

i. Miconazole Nitrate—2% (w/w)
ii. Terbinafine Hydrochloride—1% (w/w)
iii. Ketoconazole—2% (w/w)

The product used for the Stability Studies tests contained approximately 10% extra APIs (overages). It was packaged in an aluminium collapsible tube and each gram of the product contained 20.8 mg of Sodium Fusidate (in conformance with BP), which is equivalent to 20 mg of Fusidic acid (BP conformant). Detailed test results for 24 products have been presented. The % of sodium fusidate, the corticosteroid, and the antifungal used in all examples are measured w/w with respect to the final product.

### i) PRODUCT: Sodium Fusidate+Betamethasone Valerate+Miconazole Nitrate Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Betamethasone Valerate IP | 0.12% |
| iii) Miconazole Nitrate IP | 2.0% |

TABLE 3
Description Test, Batch No. SVM-01

| Conditions | Initial | 1st Month | 2nd Month | 3rd Month | 6th Month |
|---|---|---|---|---|---|
| 40° C. 75% RH | Homogenous | Homogenous | Homogenous | Homogenous | Homogenous |
| | White to | White to | White to | White to | White to |
| | off White | off White | off White | off White | off White |
| | viscous | viscous | viscous | viscous | viscous |
| | cream | cream | cream | cream | cream |
| 30° C. 65% RH | — | Do | Do | Do | Do |
| 25° C. 60% RH | — | Do | Do | Do | Do |
| Temp cycling | — | Do | — | — | — |
| Freezthaw | — | Do | — | — | — |

Measured parameter: Physical appearance
Best value of measured parameter: Homogeneous White to off White Viscous cream;
Method of measurement: Observation by naked eye

TABLE 4
pH Test, Batch No. SVM-01

| | | 1st | 2nd | 3rd | 6th |
|---|---|---|---|---|---|
| Conditions | Initial | Month | Month | Month | Month |
| 40° C. 75% RH | 4.45 | 4.44 | 4.43 | 4.44 | 4.43 |
| 30° C. 65% RH | — | 4.43 | 4.44 | 4.43 | 4.42 |
| 25° C. 60% RH | — | 4.44 | 4.43 | 4.44 | 4.43 |
| Temperature | — | 4.43 | — | — | — |
| cycling | | | | | |
| Freezthaw | — | 4.42 | — | — | — |

Measured parameter: pH;
Limits of measured parameter: 3-6
Method of measurement: Digital pH Meter

TABLE 5
Assay (%) Test, Batch No. SVM-01

| Condi- | | | 1st | 2nd | 3rd | 6th |
|---|---|---|---|---|---|---|
| tions | Assay (%) | Initial | Month | Month | Month | Month |
| 40° C. | i) Fusidic acid | 108.57 | 108.46 | 108.16 | 108.11 | 107.85 |
| 75% RH | ii) Betamethasone | 109.56 | 109.51 | 109.32 | 109.11 | 108.95 |
| | Valerate | | | | | |
| | iii) Miconazole | 107.52 | 107.44 | 107.34 | 107.22 | 107.10 |
| | Nitrate | | | | | |
| 30° C. | i) Fusidic acid | — | 108.53 | 108.41 | 108.36 | 107.99 |
| 65% RH | ii) Betamethasone | | 109.48 | 109.42 | 109.20 | 108.85 |
| | Valerate | | | | | |
| | iii) Miconazole | | 107.50 | 107.48 | 107.42 | 107.10 |
| | Nitrate | | | | | |
| 25° C. | i) Fusidic acid | — | 108.54 | 108.42 | 108.40 | 108.11 |
| 60% RH | ii) Betamethasone | | 109.54 | 109.42 | 109.21 | 109.10 |
| | Valerate | | | | | |
| | iii) Miconazole | | 107.48 | 107.44 | 107.40 | 107.30 |
| | Nitrate | | | | | |
| Temper- | i) Fusidic acid | — | 107.53 | — | — | — |
| ature | ii) Betamethasone | | 109.51 | — | — | — |
| cycling | Valerate | | | | | |
| | iii) Miconazole | | | | | |
| | Nitrate | | | | | |
| Freez- | i) Fusidic acid | — | 108.01 | — | — | — |
| thaw | ii) Betamethasone | — | 108.25 | — | — | — |
| | Valerate | | | | | |
| | iii) Miconazole | | 107.24 | — | — | — |
| | Nitrate | | | | | |

Measured parameter: Assay (%);
Limits of measured parameter: 90-110
Method of measurement: HPLC Method

### ii) PRODUCT: Sodium Fusidate+Betamethasone Valerate+Terbinafine Hydrochloride Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Betamethasone Valerate IP | 0.12% |
| iii) Terbinafine Hydrochloride BP | 1.0% |

TABLE 6
Description Test, Batch No. SVT-01

| Conditions | Initial | 1st Month | 2nd Month | 3rd Month |
|---|---|---|---|---|
| 40° C. 75% RH | Homogenous | Homog- | Homog- | Homog- |
| | White to off | enous | enous | enous |
| | White viscous | White to | White to | White to |
| | cream | off White | off White | off White |
| | | viscous | viscous | viscous |
| | | cream | cream | cream |
| 30° C. 65% RH | — | Do | Do | Do |
| 25° C. 60% RH | — | Do | Do | Do |
| Temperature | — | Do | — | — |
| cycling | | | | |
| Freezthaw | — | Do | — | — |

Measured parameter: Physical appearance
Best value of measured parameter: Homogeneous White to off White Viscous cream;
Method of measurement: Observation by naked eye

TABLE 7
pH Test, Batch No. SVT-01

| | | 1st | 2nd | 3rd |
|---|---|---|---|---|
| Conditions | Initial | Month | Month | Month |
| 40° C. 75% RH | 4.36 | 4.35 | 4.34 | 4.33 |
| 30° C. 65% RH | — | 4.34 | 4.34 | 4.33 |
| 25° C. 60% RH | — | 4.34 | 4.33 | 4.34 |
| Temperature | — | 4.33 | — | — |
| cycling | | | | |
| Freezthaw | — | 4.32 | — | — |

Measured parameter: pH;
Limits of measured parameter: 3-6
Method of measurement: Digital pH Meter

TABLE 8
Assay (%) Test, Batch No. SVT-01

| | | | 1st | 2nd | 3rd |
|---|---|---|---|---|---|
| Conditions | Assay (%) | Initial | Month | Month | Month |
| 40° C. 75% RH | i) Fusidic acid | 108.67 | 108.56 | 108.36 | 108.21 |
| | ii) Betamethasone | 109.46 | 109.41 | 109.42 | 109.22 |
| | Valerate | | | | |
| | iii) Terbinafine | 107.88 | 107.74 | 107.64 | 107.52 |
| | Hydrochloride | | | | |
| 30° C. 65% RH | i) Fusidic acid | — | 108.63 | 108.51 | 108.46 |
| | ii) Betamethasone | | 109.44 | 109.40 | 109.30 |
| | Valerate | | | | |
| | iii) Terbinafine | | 107.85 | 107.78 | 107.62 |
| | Hydrochloride | | | | |
| 25° C. 60% RH | i) Fusidic acid | — | 108.64 | 108.52 | 108.50 |
| | ii) Betamethasone | | 109.44 | 109.32 | 109.26 |
| | Valerate | | | | |
| | iii) Terbinafine | | 107.78 | 107.64 | 107.54 |
| | Hydrochloride | | | | |
| Temperature | i) Fusidic acid | — | 108.32 | — | — |
| cycling | ii) Betamethasone | | 108.51 | — | — |
| | Valerate | | | | |
| | iii) Terbinafine | | 107.21 | | |
| | Hydrochloride | | | | |
| Freezthaw | i) Fusidic acid | — | 108.18 | — | — |
| | ii) Betamethasone | — | 108.15 | — | — |
| | Valerate | | | | |
| | iii) Terbinafine | | 107.14 | — | — |
| | Hydrochloride | | | | |

Measured parameter: Assay (%)
Limits of measured parameter: 90-110
Method of measurement: HPLC Method

iii) PRODUCT: Sodium Fusidate+Betamethasone Valerate+Ketoconazole Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Betamethasone Valerate IP | 0.12% |
| iii) Ketoconazole IP | 2.0% |

TABLE 9
Description Test, Batch No. SVK-01

| Conditions | Initial | 1st Month | 2nd Month | 3rd Month |
|---|---|---|---|---|
| 40° C. 75% RH | Homogenous | Homog- | Homog- | Homog- |
| | White to off | enous | enous | enous |
| | White viscous | White to | White to | White to |
| | cream | off White | off White | off White |
| | | viscous | viscous | viscous |
| | | cream | cream | cream |
| 30° C. 65% RH | — | Do | Do | Do |
| 25° C. 60% RH | — | Do | Do | Do |
| Temperature | — | Do | — | — |
| cycling | | | | |
| Freezthaw | — | Do | — | — |

Measured parameter: Physical appearance
Best value of measured parameter: Homogeneous White to off White Viscous cream;
Method of measurement: Observation by naked eye

TABLE 10
pH Test, Batch No. SVK-01

| | | 1st | 2nd | 3rd |
|---|---|---|---|---|
| Conditions | Initial | Month | Month | Month |
| 40° C. 75% RH | 4.56 | 4.55 | 4.54 | 4.53 |
| 30° C. 65% RH | — | 4.54 | 4.54 | 4.53 |
| 25° C. 60% RH | — | 4.54 | 4.53 | 4.54 |
| Temperature | — | 4.53 | — | — |
| cycling | | | | |
| Freezthaw | — | 4.52 | — | — |

Measured parameter: pH
Limits of measured parameter: 3-6
Method of measurement: Digital pH Meter

TABLE 11
Assay (%) Test, Batch No. SVK-01

| | | | 1st | 2nd | 3rd |
|---|---|---|---|---|---|
| Conditions | Assay (%) | Initial | Month | Month | Month |
| 40° C. 75% RH | i) Fusidic acid | 108.17 | 108.16 | 108.14 | 108.11 |
| | ii) Betamethasone | 109.26 | 109.21 | 109.22 | 109.12 |
| | Valerate | | | | |
| | iii) Ketoconazole | 107.48 | 107.44 | 107.40 | 107.32 |
| 30° C. 65% RH | i) Fusidic acid | — | 108.13 | 108.11 | 108.06 |
| | ii) Betamethasone | | 109.24 | 109.20 | 109.10 |
| | Valerate | | | | |
| | iii) Ketoconazole | | 107.45 | 107.38 | 107.22 |
| 25° C. 60% RH | i) Fusidic acid | — | 108.14 | 108.12 | 108.08 |
| | ii) Betamethasone | | 109.24 | 109.22 | 109.16 |
| | Valerate | | | | |
| | iii) Ketoconazole | | 107.48 | 107.44 | 107.34 |
| Temperature | i) Fusidic acid | — | 108.12 | — | — |
| cycling | ii) Betamethasone | | 108.61 | — | — |
| | Valerate | | | | |
| | iii) Ketoconazole | | 107.81 | | |
| Freezthaw | i) Fusidic acid | — | 108.11 | — | — |
| | ii) Betamethasone | — | 108.51 | — | — |
| | Valerate | | | | |
| | iii) Ketoconazole | | 107.64 | — | — |

Measured parameter: Assay (%);
Limits of measured parameter: 90-110
Method of measurement: HPLC Method

### iv) PRODUCT: Sodium Fusidate+Fluticasone Propionate+Miconazole Nitrate Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Fluticasone Propionate BP | 0.05% |
| iii) Miconazole Nitrate IP | 2.0% |

TABLE 12
Description Test, Batch No. SFN-01

| Conditions | Initial | 1st Month | 2nd Month | 3rd Month | 6th Month |
|---|---|---|---|---|---|
| 40° C. 75% RH | Homogenous | Homogenous | Homogenous | Homogenous | Homogenous |
| | White to off | White to | White to | White to | White to |
| | White | off White | off White | off White | off White |
| | viscous | viscous | viscous | viscous | viscous |
| | cream | cream | cream | cream | cream |
| 30° C. 65% RH | — | Do | Do | Do | Do |
| 25° C. 60% RH | — | Do | Do | Do | Do |
| Temperature | — | Do | — | — | — |
| cycling | | | | | |
| Freezthaw | — | Do | — | — | — |

Measured parameter: Physical appearance
Best value of measured parameter: Homogeneous White to off White Viscous cream;
Method of measurement: Observation by naked eye

TABLE 13
pH Test, Batch No. SFN-01

| Conditions | Initial | 1st Month | 2nd Month | 3rd Month | 6th Month |
|---|---|---|---|---|---|
| 40° C. 75% RH | 3.22 | 3.21 | 3.20 | 3.21 | 3.20 |
| 30° C. 65% RH | — | 3.22 | 3.219 | 3.20 | 3.21 |
| 25° C. 60% RH | — | 3.21 | 3.21 | 3.20 | 3.19 |
| Temperature | — | 3.19 | — | — | — |
| cycling | | | | | |
| Freezthaw | — | 3.21 | — | — | — |

Measured parameter: pH
Limits of measured parameter: 3-6
Method of measurement: Digital pH Meter

TABLE 14
Assay (%) Test, Batch No. SFN-01

| Condi- | | | 1st | 2nd | 3rd | 6th |
|---|---|---|---|---|---|---|
| tions | Assay (%) | Initial | Month | Month | Month | Month |
| 40° C. | i) Fusidic acid | 108.48 | 108.46 | 108.36 | 108.28 | 108.22 |
| 75% RH | ii) Fluticasone | 108.66 | 108.55 | 108.42 | 108.31 | 108.28 |
| | Propionate | | | | | |
| | iii) Miconazole | 107.89 | 107.74 | 107.62 | 107.52 | 107.48 |
| | Nitrate | | | | | |
| | i) Fusidic acid | — | 108.43 | 108.38 | 108.34 | 108.20 |
| | ii) Fluticasone | — | 108.58 | 108.48 | 108.32 | 108.21 |
| | Propionate | | | | | |
| | iii) Miconazole | — | 107.72 | 107.68 | 107.55 | 107.48 |
| | Nitrate | | | | | |
| 25° C. | i) Fusidic acid | — | 108.44 | 108.32 | 108.28 | 108.19 |
| 60% RH | ii) Fluticasone | — | 108.64 | 108.52 | 108.41 | 108.30 |
| | Propionate | | | | | |
| | iii) Miconazole | — | 107.74 | 107.62 | 107.58 | 107.51 |
| | Nitrate | | | | | |
| Temper- | i) Fusidic acid | — | 108.12 | — | — | — |
| ature | ii) Fluticasone | — | 108.23 | — | — | — |
| cycling | Propionate | | | | | |
| | iii) Miconazole | — | 107.33 | | | |
| | Nitrate | | | | | |
| Freez- | i) Fusidic acid | — | 108.21 | — | — | — |
| thaw | ii) Fluticasone | — | 108.01 | — | — | — |
| | Propionate | | | | | |
| | iii) Miconazole | — | 107.25 | — | — | — |
| | Nitrate | | | | | |

Measured parameter: Assay (%);
Limits of measured parameter: 90-110
Method of measurement: HPLC Method

### v) PRODUCT: Sodium Fusidate+Fluticasone Propionate+Terbinafine Hydrochloride Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Fluticasone Propionate BP | 0.05% |
| iii) Terbinafine Hydrochloride BP | 1.0% |

TABLE 15
Description Test, Batch No. SFT-01

| Conditions | Initial | 1st Month | 2nd Month | 3rd Month |
|---|---|---|---|---|
| 40° C. 75% RH | Homogenous | Homogenous | Homogenous | Homogenous |
| | White to | White to | White to | White to |
| | off White | off White | off White | off White |
| | viscous | viscous | viscous | viscous |
| | cream | cream | cream | cream |
| 30° C. 65% RH | — | Do | Do | Do |
| 25° C. 60% RH | — | Do | Do | Do |
| Temperature | — | Do | — | — |
| cycling | | | | |
| Freezthaw | — | Do | — | — |

Measured parameter: Physical appearance
Best value of measured parameter: Homogeneous White to off White Viscous cream;
Method of measurement: Observation by naked eye

TABLE 16
pH Test, Batch No. SFT-01

| Conditions | Initial | 1st Month | 2nd Month | 3rd Month |
|---|---|---|---|---|
| 40° C. 75% RH | 4.28 | 4.27 | 4.26 | 4.25 |
| 30° C. 65% RH | — | 4.28 | 4.27 | 4.26 |
| 25° C. 60% RH | — | 4.27 | 4.26 | 4.26 |
| Temperature | — | 4.27 | — | — |
| cycling | | | | |
| Freezthaw | — | 4.26 | — | — |

Measured parameter: pH;
Limits of measured parameter: 3-6
Method of measurement: Digital pH Meter

TABLE 17
Assay (%) Test, Batch No. SFT-01

| | | | 1st | 2nd | 3rd |
|---|---|---|---|---|---|
| Conditions | Assay (%) | Initial | Month | Month | Month |
| 40° C. 75% | i) Fusidic acid | 109.18 | 109.16 | 109.14 | 109.08 |
| RH | ii) Fluticasone | 108.76 | 108.65 | 108.52 | 108.41 |
| | Propionate | | | | |
| | iii) Terbinafine | 107.92 | 107.84 | 107.72 | 107.62 |
| | Hydrochloride | | | | |
| 30° C. 65% | i) Fusidic acid | — | 109.13 | 109.11 | 109.10 |
| RH | ii) Fluticasone | — | 108.68 | 108.58 | 108.42 |
| | Propionate | | | | |
| | iii) Terbinafine | — | 107.85 | 107.78 | 107.65 |
| | Hydrochloride | | | | |
| 25° C. 60% | i) Fusidic acid | — | 109.17 | 109.15 | 109.11 |
| RH | ii) Fluticasone | — | 108.74 | 108.62 | 108.51 |
| | Propionate | | | | |
| | iii) Terbinafine | — | 107.75 | 107.68 | 107.62 |
| | Hydrochloride | | | | |
| Temperature | i) Fusidic acid | — | 109.12 | — | — |
| cycling | ii) Fluticasone | — | 108.43 | — | — |
| | Propionate | | | | |
| | iii) Terbinafine | — | 107.43 | | |
| | Hydrochloride | | | | |
| Freezthaw | i) Fusidic acid | — | 108.91 | — | — |
| | ii) Fluticasone | — | 108.11 | — | — |
| | Propionate | | | | |
| | iii) Terbinafine | — | 107.52 | — | — |
| | Hydrochloride | | | | |

Measured parameter: Assay (%);
Limits of measured parameter: 90-110
Method of measurement: HPLC Method

### vi) PRODUCT: Sodium Fusidate+Fluticasone Propionate+Ketoconazole Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Fluticasone Propionate BP | 0.05% |
| iii) Ketoconazole IP | 2.0% |

TABLE 18
Description Test, Batch No. SFK-01

| Conditions | Initial | 1st Month | 2nd Month | 3rd Month |
|---|---|---|---|---|
| 40° C. 75% RH | Homogenous | Homogenous | Homogenous | Homogenous |
| | White to | White to | White to | White to |
| | off White | off White | off White | off White |
| | viscous | viscous | viscous | viscous |
| | cream | cream | cream | cream |
| 30° C. 65% RH | — | Do | Do | Do |
| 25° C. 60% RH | — | Do | Do | Do |
| Temperature | — | Do | — | — |
| cycling | | | | |
| Freezthaw | — | Do | — | — |

Measured parameter: Physical appearance
Best value of measured parameter: Homogeneous White to off White Viscous cream;
Method of measurement: Observation by naked eye

TABLE 19
pH Test, Batch No. SFK-01

| Conditions | Initial | 1st Month | 2nd Month | 3rd Month |
|---|---|---|---|---|
| 40° C. 75% RH | 4.61 | 4.61 | 4.60 | 4.59 |
| 30° C. 65% RH | — | 4.60 | 4.59 | 4.58 |
| 25° C. 60% RH | — | 4.61 | 4.60 | 4.60 |
| Temperature | — | 4.60 | — | — |
| cycling | | | | |
| Freezthaw | — | 4.61 | — | — |

Measured parameter: pH;
Limits of measured parameter: 3-6
Method of measurement: Digital pH Meter

TABLE 20
Assay (%) Test, Batch No. SFK-01

| | | | 1st | 2nd | 3rd |
|---|---|---|---|---|---|
| Conditions | Assay (%) | Initial | Month | Month | Month |
| 40° C. 75% | i) Fusidic acid | 109.28 | 109.26 | 109.24 | 109.18 |
| RH | ii) Fluticasone | 108.86 | 108.75 | 108.62 | 108.51 |
| | Propionate | | | | |
| | iii) Ketoconazole | 107.62 | 107.54 | 107.52 | 107.42 |
| 30° C. 65% | i) Fusidic acid | — | 109.23 | 109.21 | 109.15 |
| RH | ii) Fluticasone | — | 108.78 | 108.68 | 108.54 |
| | Propionate | | | | |
| | iii) Ketoconazole | — | 107.60 | 107.58 | 107.55 |
| 25° C. 60% | i) Fusidic acid | — | 109.27 | 109.25 | 109.18 |
| RH | ii) Fluticasone | — | 108.77 | 108.68 | 108.61 |
| | Propionate | | | | |
| | iii) Ketoconazole | — | 107.60 | 107.54 | 107.48 |
| Temperature | i) Fusidic acid | — | 109.22 | — | — |
| cycling | ii) Fluticasone | — | 108.53 | — | — |
| | Propionate | | | | |
| | iii) Ketoconazole | — | 107.23 | | |
| Freezthaw | i) Fusidic acid | — | 108.41 | — | — |
| | ii) Fluticasone | — | 107.11 | — | — |
| | Propionate | | | | |
| | iii) Ketoconazole | — | 107.12 | — | — |

Measured parameter: Assay (%);
Limits of measured parameter: 90-110
Method of measurement: HPLC Method

vii) PRODUCT: Sodium Fusidate+Mometasone Furoate+Miconazole Nitrate Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Mometasone Furoate USP | 0.1% |
| iii) Miconazole Nitrate IP | 2.0% |

TABLE 21
Description Test, Batch No. SMN-01

| | | 1st | 2nd | 3rd | 6th |
|---|---|---|---|---|---|
| Conditions | Initial | Month | Month | Month | Month |
| 40° C. 75% RH | Homogenous | Homogenous | Homogenous | Homogenous | Homogenous |
| | White to | White to | White | White to | White |
| | off White | off | to off | off | to off |
| | viscous | White | White | White | White |
| | cream | viscous | viscous | viscous | viscous |
| | | cream | cream | cream | cream |
| 30° C. 65% RH | — | Do | Do | Do | Do |
| 25° C. 60% RH | — | Do | Do | Do | Do |
| Temperature | — | Do | — | — | — |
| cycling | | | | | |
| Freezthaw | — | Do | — | — | — |

Measured parameter: Physical appearance
Best value of measured parameter: Homogeneous White to off White Viscous cream;
Method of measurement: Observation by naked eye

TABLE 22
pH Test, Batch No. SMN-01

| Conditions | Initial | 1st Month | 2nd Month | 3rd Month | 6th Month |
|---|---|---|---|---|---|
| 40° C. 75% RH | 3.64 | 3.63 | 3.62 | 3.63 | 3.62 |
| 30° C. 65% RH | — | 3.62 | 3.63 | 3.64 | 3.63 |
| 25° C. 60% RH | — | 3.63 | 3.64 | 3.63 | 3.62 |
| Temperature | — | 3.62 | — | — | — |
| cycling | | | | | |
| Freezthaw | — | 3.63 | — | — | — |

Measured parameter: pH;
Limits of measured parameter: 3-6
Method of measurement: Digital pH Meter

TABLE 23
Assay (%) Test, Batch No. SMN-01

| Condi- | | | 1st | 2nd | 3rd | 6th |
|---|---|---|---|---|---|---|
| tions | Assay (%) | Initial | Month | Month | Month | Month |
| 40° C. | i) Fusidic acid | 108.37 | 108.36 | 108.24 | 108.18 | 107.79 |
| 75% RH | ii) Mometasone | 108.56 | 108.51 | 108.32 | 108.11 | 107.88 |
| | Furoate | | | | | |
| | iii) Miconazole | 107.88 | 107.78 | 107.68 | 107.44 | 107.34 |
| | Nitrate | | | | | |
| 30° C. | i) Fusidic acid | — | 108.33 | 108.31 | 108.26 | 108.12 |
| 65% RH | ii) Mometasone | — | 108.52 | 108.40 | 108.32 | 108.24 |
| | Furoate | | | | | |
| | iii) Miconazole | — | 107.81 | 107.78 | 107.54 | 107.34 |
| | Nitrate | | | | | |
| 25° C. | i) Fusidic acid | — | 108.24 | 108.22 | 108.20 | 107.95 |
| 60% RH | ii) Mometasone | — | 108.54 | 108.42 | 108.21 | 107.82 |
| | Furoate | | | | | |
| | iii) Miconazole | — | 107.74 | 107.64 | 107.52 | 107.26 |
| | Nitrate | | | | | |
| Temper- | i) Fusidic acid | — | 107.63 | — | — | — |
| ature | ii) Mometasone | — | 108.51 | — | — | — |
| cycling | Furoate | | | | | |
| | iii) Miconazole | — | 107.35 | | | |
| | Nitrate | | | | | |
| Freez- | i) Fusidic acid | — | 108.11 | — | — | — |
| thaw | ii) Mometasone | — | 108.15 | — | — | — |
| | Furoate | | | | | |
| | iii) Miconazole | — | 107.25 | — | — | — |
| | Nitrate | | | | | |

Measured parameter: Assay (%)
Limits of measured parameter: 90-110
Method of measurement: HPLC Method

vii) PRODUCT: Sodium Fusidate+Mometasone Furoate+Terbinafine Hydrochloride Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Mometasone Furoate USP | 0.1% |
| iii) Terbinafine Hydrochloride BP | 1.0% |

TABLE 24
Description Test, Batch No. SMT-01

| Conditions | Initial | 1st Month | 2nd Month | 3rd Month |
|---|---|---|---|---|
| 40° C. 75% RH | Homogenous | Homogenous | Homogenous | Homogenous |
| | White to | White to | White to | White to |
| | off White | off White | off White | off White |
| | viscous | viscous | viscous | viscous |
| | cream | cream | cream | cream |
| 30° C. 65% RH | — | Do | Do | Do |
| 25° C. 60% RH | — | Do | Do | Do |
| Temp cycling | — | Do | — | — |
| Freezthaw | — | Do | — | — |

Measured parameter: Physical appearance
Best value of measured parameter: Homogeneous White to off White Viscous cream;
Method of measurement: Observation by naked eye

TABLE 25
Assay (%) Test, Batch No. SMT-01

| | | | 1st | 2nd | 3rd |
|---|---|---|---|---|---|
| Conditions | Assay (%) | Initial | Month | Month | Month |
| 40° C. 75% | i) Fusidic acid | 109.37 | 109.36 | 109.24 | 109.18 |
| RH | ii) Mometasone Furoate | 107.56 | 107.51 | 107.32 | 107.11 |
| | iii) Terbinafine | 108.88 | 108.78 | 108.68 | 108.44 |
| | Hydrochloride | | | | |
| 30° C. 65% | i) Fusidic acid | — | 109.33 | 109.31 | 109.26 |
| RH | ii) Mometasone Furoate | — | 107.52 | 107.40 | 107.32 |
| | iii) Terbinafine | — | 108.81 | 108.78 | 108.54 |
| | Hydrochloride | | | | |
| 25° C. 60% | i) Fusidic acid | — | 109.24 | 109.22 | 109.20 |
| RH | ii) Mometasone Furoate | — | 107.54 | 107.42 | 107.21 |
| | iii) Terbinafine | — | 108.74 | 108.64 | 108.52 |
| | Hydrochloride | | | | |
| Temperature | i) Fusidic acid | — | 108.63 | — | — |
| cycling | ii) Mometasone Furoate | — | 107.51 | — | — |
| | iii) Terbinafine | — | 108.35 | | |
| | Hydrochloride | | | | |
| Freezthaw | i) Fusidic acid | — | 109.11 | — | — |
| | ii) Mometasone Furoate | — | 107.15 | — | — |
| | iii) Terbinafine | — | 108.25 | — | — |
| | Hydrochloride | | | | |

Measured parameter: Assay (%);
Limits of measured parameter: 90-110
Method of measurement: HPLC Method

TABLE 26
pH Test, Batch No. SMT-01

| Conditions | Initial | 1st Month | 2nd Month | 3rd Month |
|---|---|---|---|---|
| 40° C. 75% RH | 3.96 | 3.95 | 3.94 | 3.94 |
| 30° C. 65% RH | — | 3.96 | 3.95 | 3.94 |
| 25° C. 60% RH | — | 3.95 | 3.94 | 9.94 |
| Temperature | — | 3.95 | — | — |
| cycling | | | | |
| Freezthaw | — | 3.94 | — | — |

Measured parameter: pH;
Limits of measured parameter: 3-6
Method of measurement: Digital pH Meter

### ix) PRODUCT: Sodium Fusidate+Mometasone Furoate+Ketoconazole Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Mometasone Furoate USP | 0.1% |
| iii) Ketoconazole IP | 2.0% |

TABLE 27
Description Test, Batch No. SMK-01

| Conditions | Initial | 1st Month | 2nd Month | 3rd Month |
|---|---|---|---|---|
| 40° C. 75% RH | Homogenous | Homogenous | Homogenous | Homogenous |
| | White to | White to | White to | White to |
| | off White | off White | off White | off White |
| | viscous | viscous | viscous | viscous |
| | cream | cream | cream | cream |
| 30° C. 65% RH | — | Do | Do | Do |
| 25° C. 60% RH | — | Do | Do | Do |
| Temperature | — | Do | — | — |
| cycling | | | | |
| Freezthaw | — | Do | — | — |

Measured parameter: Physical appearance
Best value of measured parameter: Homogeneous White to off White Viscous cream;
Method of measurement: Observation by naked eye

TABLE 28
pH Test, Batch No. SMK-01

| Conditions | Initial | 1st Month | 2nd Month | 3rd Month |
|---|---|---|---|---|
| 40° C. 75% RH | 3.91 | 3.90 | 3.90 | 3.89 |
| 30° C. 65% RH | — | 3.91 | 3.90 | 3.89 |
| 25° C. 60% RH | — | 3.91 | 3.90 | 3.90 |
| Temperature | — | 3.90 | — | — |
| cycling | | | | |
| Freezthaw | — | 3.89 | — | — |

Measured parameter: pH;
Limits of measured parameter: 3-6
Method of measurement: Digital pH Meter

TABLE 29
Assay (%) Test, Batch No. SMK-01

| | | | 1st | 2nd | 3rd |
|---|---|---|---|---|---|
| Conditions | Assay (%) | Initial | Month | Month | Month |
| 40° C. 75% | i) Fusidic acid | 108.47 | 108.36 | 108.24 | 108.18 |
| RH | ii) Mometasone Furoate | 107.46 | 107.41 | 107.42 | 107.21 |
| | iii) Ketoconazole | 107.48 | 107.38 | 107.28 | 107.14 |
| 30° C. 65% | i) Fusidic acid | — | 108.33 | 108.31 | 108.26 |
| RH | ii) Mometasone Furoate | — | 107.42 | 107.34 | 107.22 |
| | iii) Ketoconazole | — | 107.38 | 107.28 | 107.22 |
| 25° C. 60% | i) Fusidic acid | — | 108.24 | 108.22 | 108.20 |
| RH | ii) Mometasone Furoate | — | 107.44 | 107.41 | 107.34 |
| | iii) Ketoconazole | — | 107.45 | 107.34 | 107.32 |
| Temperature | i) Fusidic acid | — | 108.43 | — | — |
| cycling | ii) Mometasone Furoate | — | 107.41 | — | — |
| | iii) Ketoconazole | — | 107.35 | | |
| Freezthaw | i) Fusidic acid | — | 108.11 | — | — |
| | ii) Mometasone Furoate | — | 107.25 | — | — |
| | iii) Ketoconazole | — | 107.35 | — | — |

Measured parameter: Assay (%);
limits of measured parameter: 90-110
Method of measurement: HPLC Method

x) PRODUCT: Sodium Fusidate+Dexamethasone Acetate+Miconazole Nitrate Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Dexamethasone Acetate IP | 0.1% |
| iii) Miconazole Nitrate IP | 2.0% |

TABLE 30
Description Test, Batch No. SDM-01

| Conditions | Initial | 1st Month | 2nd Month | 3rd Month |
|---|---|---|---|---|
| 40° C. 75% RH | Homogenous | Homogenous | Homogenous | Homogenous |
| | White to off White | White to off White | White to off | White to off |
| | viscous cream | viscous cream | White viscous | White viscous |
| | | | cream | cream |
| 30° C. 65% RH | — | Do | Do | Do |
| 25° C. 60% RH | — | Do | Do | Do |
| Temperature | — | Do | — | — |
| cycling | | | | |
| Freezthaw | — | Do | — | — |

Measured parameter: Physical appearance
Best value of measured parameter: Homogeneous White to off White Viscous cream;
Method of measurement: Observation by naked eye

TABLE 31
pH Test, Batch No. SDM-01

| Conditions | Initial | 1st Month | 2nd Month | 3rd Month |
|---|---|---|---|---|
| 40° C. 75% RH | 4.36 | 4.36 | 4.35 | 4.34 |
| 30° C. 65% RH | — | 4.35 | 4.35 | 4.34 |
| 25° C. 60% RH | — | 4.36 | 4.35 | 4.34 |
| Temperature | — | 4.34 | — | — |
| cycling | | | | |
| Freezthaw | — | 4.35 | — | — |

Measured parameter: pH;
Limits of measured parameter: 3-6
Method of measurement: Digital pH Meter

TABLE 32
Assay (%) Test, Batch No. SDM-01

| | | | 1st | 2nd | 3rd |
|---|---|---|---|---|---|
| Conditions | Assay (%) | Initial | Month | Month | Month |
| 40° C. 75% | i) Fusidic acid | 109.62 | 109.58 | 109.44 | 109.30 |
| RH | ii) Dexamethasone | 108.25 | 108.24 | 108.22 | 108.15 |
| | Acetate | | | | |
| | iii) Miconazole | 108.15 | 108.11 | 108.05 | 107.89 |
| | Nitrate | | | | |
| 30° C. 65% | i) Fusidic acid | — | 109.63 | 109.52 | 109.32 |
| RH | ii) Dexamethasone | — | 108.24 | 108.22 | 108.19 |
| | Acetate | | | | |
| | iii) Miconazole | — | 108.14 | 108.11 | 108.02 |
| | Nitrate | | | | |
| 25° C. 60% | i) Fusidic acid | — | 109.50 | 109.34 | 109.26 |
| RH | ii) Dexamethasone | — | 108.24 | 108.11 | 108.05 |
| | Acetate | | | | |
| | iii) Miconazole | — | 108.11 | 108.08 | 108.03 |
| | Nitrate | | | | |
| Temperature | i) Fusidic acid | — | 109.52 | — | — |
| cycling | ii) Dexamethasone | — | 108.12 | — | — |
| | Acetate | | | | |
| | iii) Miconazole | — | 107.86 | | |
| | Nitrate | | | | |
| Freezthaw | i) Fusidic acid | — | 108.91 | — | — |
| | ii) Dexamethasone | — | 107.94 | — | — |
| | Acetate | | | | |
| | iii) Miconazole | — | 107.94 | | |
| | Nitrate | | | | |

Measured parameter: Assay (%);
Limits of measured parameter: 90-110
Method of measurement: HPLC Method

### Xi) PRODUCT: Sodium Fusidate+Dexamethasone Acetate+Terbinafine Hydrochloride Cream

### PACK: Aluminum Collapsible Tube

| Composition: | Each gm contains: |
|---|---|
| i) Sodium Fusidate BP Equivalent | 2.0% |
| to Fusidic Acid BP | |
| ii) Dexamethasone Acetate IP | 0.1% |
| iii) Terbinafine Hydrochloride BP | 1.0% |

TABLE 33
Description Test, Batch No. SDT-01

| Conditions | Initial | 1st Month | 2nd Month | 3rd Month |
|---|---|---|---|---|
| 40° C. 75% RH | Homogenous | Homogenous | Homogenous | Homogenous |
| | White to off | White to off | White to off | White to off |
| | White viscous cream | White viscous cream | White viscous | White viscous |
| | | | cream | cream |
| 30° C. 65% RH | — | Do | Do | Do |
| 25° C. 60% RH | — | Do | Do | Do |
| Temp cycling | — | Do | — | — |
| Freezthaw | — | Do | — | — |

Measured parameter: Physical appearance
Best value of measured parameter: Homogeneous White to off White Viscous cream;
Method of measurement: Observation by naked eye

TABLE 34
Assay (%) Test, Batch No. SDT-01

| | | | 1st | 2nd | 3rd |
|---|---|---|---|---|---|
| Conditions | Assay (%) | Initial | Month | Month | Month |
| 40° C. 75% | i) Fusidic acid | 108.66 | 108.59 | 108.48 | 108.35 |
| RH | ii) Dexamethasone | 108.35 | 108.34 | 108.32 | 108.25 |
| | Acetate | | | | |
| | iii) Terbinafine | 107.15 | 107.11 | 107.05 | 107.01 |
| | Hydrochloride | | | | |
| 30° C. 65% | i) Fusidic acid | — | 108.63 | 108.52 | 108.32 |
| RH | ii) Dexamethasone | — | 108.34 | 108.32 | 108.29 |
| | Acetate | | | | |
| | iii) Terbinafine | — | 107.14 | 107.11 | 107.02 |
| | Hydrochloride | | | | |
| 25° C. 60% | i) Fusidic acid | — | 108.51 | 108.36 | 108.28 |
| RH | ii) Dexamethasone | — | 108.24 | 108.21 | 108.15 |
| | Acetate | | | | |
| | iii) Terbinafine | — | 107.12 | 107.08 | 107.03 |
| | Hydrochloride | | | | |
| Temperature | i) Fusidic acid | — | 108.52 | — | — |
| cycling | ii) Dexamethasone | — | 108.12 | — | — |
| | Acetate | | | | |
| | iii) Terbinafine | — | 107.08 | | |
| | Hydrochloride | | | | |
| Freezthaw | i) Fusidic acid | — | 108.11 | — | — |
| | ii) Dexamethasone | — | 108.21 | — | — |
| | Acetate | | | | |
| | iii) Terbinafine | — | 107.11 | — | — |
| | Hydrochloride | | | | |

Measured parameter: Assay (%);
Limits of measured parameter: 90-110
Method of measurement: HPLC Method

TABLE 35
pH Test, Batch No. SDT-01

| Conditions | Initial | 1st Month | 2nd Month | 3rd Month |
|---|---|---|---|---|
| 40° C. 75% RH | 4.51 | 4.50 | 4.50 | 4.49 |
| 30° C. 65% RH | — | 4.51 | 4.50 | 4.50 |
| 25° C. 60% RH | — | 4.50 | 4.50 | 4.49 |
| Temp cycling | — | 4.51 | — | — |
| Freezthaw | — | 4.50 | — | — |

Measured parameter: pH;
Limits of measured parameter: 3-6
Method of measurement: Digital pH Meter

xii) PRODUCT: Sodium Fusidate+Dexamethasone Acetate+Ketoconazole Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Dexamethasone Acetate | IP | 0.1% |
| iii) Ketoconazole | IP | 2.0% |

TABLE 36
Description Test, Batch No. SDK-01
Measured parameter: Physical appearance
Best value of measured parameter:
Homogeneous White to off White Viscous cream;
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to off | White | White | White |
| | White | to off | to off | to off |
| | viscous | White | White | White |
| | cream | viscous | viscous | viscous |
| | | cream | cream | cream |
| 30° C. | — | Homogenous | Homogenous | Homogenous |
| 65% RH | | White | White | White |
| | | to off | to off | to off |
| | | White | White | White |
| | | viscous | viscous | viscous |
| | | cream | cream | cream |
| 25° C. | — | Homogenous | Homogenous | Homogenous |
| 60% RH | | White | White | White |
| | | to off | to off | to off |
| | | White | White | White |
| | | viscous | viscous | viscous |
| | | cream | cream | cream |
| Temp cycling | — | Homogenous | — | — |
| | | White | | |
| | | to off | | |
| | | White | | |
| | | viscous | | |
| | | cream | | |
| Freezthaw | — | Homogenous | — | — |
| | | White | | |
| | | to off | | |
| | | White | | |
| | | viscous | | |
| | | cream | | |

TABLE 37
pH Test, Batch No. SDK-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.46 | 4.45 | 4.45 | 4.44 |
| 30° C. 65% RH | — | 4.46 | 4.45 | 4.45 |
| 25° C. 60% RH | — | 4.45 | 4.44 | 4.44 |
| Temperature | — | 4.45 | — | — |
| cycling | | | | |
| Freezthaw | — | 4.44 | — | — |

TABLE 38
Assay (%) Test, Batch No. SDK-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 107.66 | 107.59 | 107.48 | 107.35 |
| RH | ii) Dexamethasone | 108.65 | 108.54 | 108.42 | 108.35 |
| | Acetate | | | | |
| | iii) Ketoconazole | 107.55 | 107.45 | 107.38 | 107.31 |
| 30° C. 65% | i) Fusidic acid | — | 107.63 | 107.52 | 107.32 |
| RH | ii) Dexamethasone | — | 108.64 | 108.52 | 108.39 |
| | Acetate | | | | |
| | iii) Ketoconazole | — | 107.54 | 107.41 | 107.35 |
| 25° C. 60% | i) Fusidic acid | — | 107.51 | 107.36 | 107.28 |
| RH | ii) Dexamethasone | — | 107.54 | 107.41 | 107.35 |
| | Acetate | | | | |
| | iii) Ketoconazole | — | 107.41 | 107.39 | 107.30 |
| Temperature | i) Fusidic acid | — | 107.52 | — | — |
| cycling | ii) Dexamethasone | — | 108.22 | — | — |
| | Acetate | | | | |
| | iii) Ketoconazole | — | 107.45 | | |
| Freezthaw | i) Fusidic acid | — | 107.21 | — | — |
| | ii) Dexamethasone | — | 108.25 | — | — |
| | Acetate | | | | |
| | iii) Ketoconazole | — | 107.31 | — | — |

xii) PRODUCT: Sodium Fusidate+Hydrocortisone Acetate+Miconazole Nitrate Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Hydrocortisone Acetate | IP | 1.0% |
| iii) Miconazole Nitrate | IP | 2.0% |

TABLE 39
Description Test, Batch No. HSM-01
Measured parameter: Physical appearance
Best value of measured parameter:
Homogeneous White to off White Viscous cream;
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to | White to | White to | White to |
| | off White | off White | off White | off White |
| | viscous | viscous | viscous | viscous |
| | cream | cream | cream | cream |
| 30° C. | — | Homogenous | Homogenous | Homogenous |
| 65% RH | | White to | White to | White to |
| | | off White | off White | off White |
| | | viscous | viscous | viscous |
| | | cream | cream | cream |
| 25° C. | — | Homogenous | Homogenous | Homogenous |
| 60% RH | | White to | White to | White to |
| | | off White | off White | off White |
| | | viscous | viscous | viscous |
| | | cream | cream | cream |
| Temp cycling | — | Homogenous | — | — |
| | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 40
pH Test, Batch No. HSM-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.36 | 4.35 | 4.35 | 4.34 |
| 30° C. 65% RH | — | 4.36 | 4.35 | 4.35 |
| 25° C. 60% RH | — | 4.35 | 4.34 | 4.34 |
| Temperature | — | 4.35 | — | — |
| cycling | | | | |
| Freezthaw | — | 4.34 | — | — |

TABLE 41
Assay (%) Test, Batch No. HSM-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 108.58 | 108.45 | 108.38 | 108.28 |
| RH | ii) Hydrocortisone | 107.65 | 107.54 | 107.48 | 107.35 |
| | Acetate | | | | |
| | iii) Miconazole | 108.25 | 108.22 | 108.12 | 108.08 |
| | Nitrate | | | | |
| 30° C. 65% | i) Fusidic acid | — | 108.54 | 108.40 | 108.30 |
| RH | ii) Hydrocortisone | — | 107.64 | 107.52 | 107.39 |
| | Acetate | | | | |
| | iii) Miconazole | — | 108.20 | 108.15 | 108.10 |
| | Nitrate | | | | |
| 25° C. 60% | i) Fusidic acid | — | 108.52 | 108.41 | 108.32 |
| RH | ii) Hydrocortisone | — | 107.54 | 107.31 | 107.28 |
| | Acetate | | | | |
| | iii) Miconazole | — | 108.22 | 108.19 | 108.14 |
| | Nitrate | | | | |
| Temperature | i) Fusidic acid | — | 108.40 | — | — |
| cycling | ii) Hydrocortisone | | 107.11 | — | — |
| | Acetate | | | | |
| | iii) Miconazole | — | 108.22 | | |
| | Nitrate | | | | |
| Freezthaw | i) Fusidic acid | — | 108.31 | — | — |
| | ii) Hydrocortisone | — | 107.14 | — | — |
| | Acetate | | | | |
| | iii) Miconazole | — | 108.14 | — | — |
| | Nitrate | | | | |

xiv) PRODUCT: Sodium Fusidate+Hydrocortisone Acetate+Terbinafine Hydrochloride Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Hydrocortisone Acetate | IP | 1.0% |
| iii) Terbinafine Hydrochloride | BP | 1.0% |

TABLE 42
Description Test, Batch No. HST-01
Measured parameter: Physical appearance
Best value of measured parameter:
Homogeneous White to off White Viscous cream;
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to off | White to | White to | White to off |
| | White | off White | off White | White |
| | viscous | viscous | viscous | viscous |
| | cream | cream | cream | cream |
| 30° C. | — | Homogenous | Homogenous | Homogenous |
| 65% RH | | White to | White to | White to off |
| | | off White | off White | White |
| | | viscous | viscous | viscous |
| | | cream | cream | cream |
| 25° C. | — | Homogenous | Homogenous | Homogenous |
| 60% RH | | White to | White to | White to off |
| | | off White | off White | White |
| | | viscous | viscous | viscous |
| | | cream | cream | cream |
| Temp cycling | — | Homogenous | — | — |
| | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 43
Assay (%) Test, Batch No. HST-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. | i) Fusidic acid | 108.88 | 108.85 | 108.78 | 108.68 |
| 75% RH | ii) Hydrocortisone | 108.65 | 108.54 | 108.48 | 108.35 |
| | Acetate | | | | |
| | iii) Terbinafine | 107.25 | 107.22 | 107.12 | 107.08 |
| | Hydrochloride | | | | |
| 30° C. | i) Fusidic acid | — | 108.84 | 108.74 | 108.68 |
| 65% RH | ii) Hydrocortisone | — | 108.64 | 108.52 | 108.39 |
| | Acetate | | | | |
| | iii) Terbinafine | — | 107.20 | 107.15 | 107.10 |
| | Hydrochloride | | | | |
| 25° C. | i) Fusidic acid | — | 108.82 | 108.74 | 108.72 |
| 60% RH | ii) Hydrocortisone | — | 108.54 | 108.31 | 108.28 |
| | Acetate | | | | |
| | iii) Terbinafine | — | 107.22 | 107.19 | 107.14 |
| | Hydrochloride | | | | |
| Temperature | i) Fusidic acid | — | 108.48 | — | — |
| cycling | ii) Hydrocortisone | | 108.11 | — | — |
| | Acetate | | | | |
| | iii) Terbinafine | — | 107.22 | | |
| | Hydrochloride | | | | |
| Freezthaw | i) Fusidic acid | — | 108.45 | — | — |
| | ii) Hydrocortisone | — | 108.14 | — | — |
| | Acetate | | | | |
| | iii) Terbinafine | — | 107.14 | — | — |
| | Hydrochloride | | | | |

TABLE 44
pH Test, Batch No. HST-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.32 | 4.32 | 4.31 | 4.30 |
| 30° C. 65% RH | — | 4.32 | 4.31 | 4.30 |
| 25° C. 60% RH | — | 4.31 | 4.31 | 4.30 |
| Temp cycling | — | 4.32 | — | — |
| Freezthaw | — | 4.31 | — | — |

### xv) PRODUCT: Sodium Fusidate+Hydrocortisone Acetate+Ketoconazole Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Hydrocortisone Acetate | IP | 1.0% |
| iii) Ketoconazole | IP | 2.0% |

TABLE 45
Description Test, Batch No. HSK-01
Measured parameter: Physical appearance
Best value of measured parameter:
Homogeneous White to off White Viscous cream;
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to off | White to | White to off | White to off |
| | White | off White | White viscous | White |
| | viscous | viscous | cream | viscous |
| | cream | cream | | cream |
| 30° C. | — | Homogenous | Homogenous | Homogenous |
| 65% RH | | White to | White to off | White to off |
| | | off White | White viscous | White |
| | | viscous | cream | viscous |
| | | cream | | cream |
| 25° C. | — | Homogenous | Homogenous | Homogenous |
| 60% RH | | White to | White to off | White to off |
| | | off White | White viscous | White |
| | | viscous | cream | viscous |
| | | cream | | cream |
| Temp | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 46
pH Test, Batch No. HSK-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.51 | 4.50 | 4.50 | 4.49 |
| 30° C. 65% RH | — | 4.50 | 4.49 | 4.49 |
| 25° C. 60% RH | — | 4.51 | 4.50 | 4.49 |
| Temperature | — | 4.49 | — | — |
| cycling | | | | |
| Freezthaw | — | 4.50 | — | — |

TABLE 47
Assay (%) Test, Batch No. HSK-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 107.68 | 107.65 | 107.58 | 107.48 |
| RH | ii) Hydrocortisone | 107.85 | 107.81 | 107.78 | 107.55 |
| | Acetate | | | | |
| | iii) Ketoconazole | 107.52 | 107.42 | 107.32 | 107.28 |
| 30° C. 65% | i) Fusidic acid | — | 107.64 | 107.54 | 107.48 |
| RH | ii) Hydrocortisone | — | 107.74 | 107.65 | 107.49 |
| | Acetate | | | | |
| | iii) Ketoconazole | — | 107.42 | 107.31 | 107.22 |
| 25° C. 60% | i) Fusidic acid | — | 107.62 | 107.54 | 107.42 |
| RH | ii) Hydrocortisone | — | 107.54 | 107.31 | 107.28 |
| | Acetate | | | | |
| | iii) Ketoconazole | — | 107.42 | 107.25 | 107.12 |
| Temperature | i) Fusidic acid | — | 107.48 | — | — |
| cycling | ii) Hydrocortisone | | 107.15 | — | — |
| | Acetate | | | | |
| | iii) Ketoconazole | — | 107.42 | | |
| Freezthaw | i) Fusidic acid | — | 107.35 | — | — |
| | ii) Hydrocortisone | — | 107.14 | — | — |
| | Acetate | | | | |
| | iii) Ketoconazole | — | 107.24 | — | — |

xvi) PRODUCT: Sodium Fusidate+Clobetasol Propionate+Miconazole Nitrate Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Clobetasol Propionate | USP | 0.05% |
| iii) Miconazole Nitrate | IP | 2.0% |

TABLE 48
Description Test, Batch No. SCM-01
Measured parameter: Physical appearance
Best value of measured parameter:
Homogeneous White to off White Viscous cream;
Method of measurement: Observation by naked eye

| | | 1st | 2nd | 3rd | 6th |
|---|---|---|---|---|---|
| Conditions | Initial | Month | Month | Month | Month |
| 40° C. | Homog- | Homog- | Homog- | Homog- | Homog- |
| 75% RH | enous | enous | enous | enous | enous |
| | White to | White to | White to | White to | White to |
| | off White | off White | off White | off White | off White |
| | viscous | viscous | viscous | viscous | viscous |
| | cream | cream | cream | cream | cream |
| 30° C. | — | Homog- | Homog- | Homog- | Homog- |
| 65% RH | | enous | enous | enous | enous |
| | | White to | White to | White to | White to |
| | | off White | off White | off White | off White |
| | | viscous | viscous | viscous | viscous |
| | | cream | cream | cream | cream |
| 25° C. | — | Homog- | Homog- | Homog- | Homog- |
| 60% RH | | enous | enous | enous | enous |
| | | White to | White to | White to | White to |
| | | off White | off White | off White | off White |
| | | viscous | viscous | viscous | viscous |
| | | cream | cream | cream | cream |
| Temperature | — | Homog- | — | — | — |
| cycling | | enous | | | |
| | | White to | | | |
| | | off White | | | |
| | | viscous | | | |
| | | cream | | | |
| Freezthaw | — | Homog- | — | — | — |
| | | enous | | | |
| | | White to | | | |
| | | off White | | | |
| | | viscous | | | |
| | | cream | | | |

TABLE 49
pH Test, Batch No. SCM-01
Measured parameter: pH; Limits of measured parameter: 3-6
Method of measurement: Digital pH Meter

| | | | | | 6th |
|---|---|---|---|---|---|
| Conditions | Initial | 1st Month | 2nd Month | 3rd Month | Month |
| 40° C. 75% RH | 4.42 | 4.41 | 4.40 | 4.40 | 4.39 |
| 30° C. 65% RH | — | 4.42 | 4.41 | 4.40 | 4.39 |
| 25° C. 60% RH | — | 4.42 | 4.41 | 4.41 | 4.40 |
| Temperature | — | 4.40 | — | — | — |
| cycling | | | | | |
| Freezthaw | — | 4.41 | — | — | — |

TABLE 50
Assay (%) Test, Batch No. SCM-01

| Condi- | | | 1st | 2nd | 3rd | 6th |
|---|---|---|---|---|---|---|
| tions | Assay (%) | Initial | Month | Month | Month | Month |
| 40° C. | i) Fusidic acid | 108.48 | 108.43 | 108.34 | 108.28 | 108.15 |
| 75% RH | ii) Clobetasol | 108.41 | 108.34 | 108.22 | 108.15 | 108.10 |
| | Propionate | | | | | |
| | iii) Miconazole | 107.85 | 107.78 | 107.69 | 107.54 | 107.45 |
| | Nitrate | | | | | |
| 30° C. | i) Fusidic acid | — | 108.41 | 108.38 | 108.32 | 108.21 |
| 65% RH | ii) Clobetasol | — | 108.38 | 108.32 | 108.29 | 108.22 |
| | Propionate | | | | | |
| | iii) Miconazole | — | 107.82 | 107.79 | 107.64 | 107.55 |
| | Nitrate | | | | | |
| 25° C. | i) Fusidic acid | — | 108.42 | 108.34 | 108.25 | 108.18 |
| 60% RH | ii) Clobetasol | — | 108.40 | 108.35 | 108.25 | 108.18 |
| | Propionate | | | | | |
| | iii) Miconazole | — | 107.84 | 107.81 | 107.74 | 107.65 |
| | Nitrate | | | | | |
| Temper- | i) Fusidic acid | — | 108.38 | — | — | — |
| ature | ii) Clobetasol | — | 108.21 | — | — | — |
| cycling | Propionate | | | | | |
| | iii) Miconazole | | 107.62 | | | |
| | Nitrate | | | | | |
| Freez- | i) Fusidic acid | — | 108.32 | — | — | — |
| thaw | ii) Clobetasol | — | 108.11 | — | — | — |
| | Propionate | | | | | |
| | iii) Miconazole | — | 107.26 | — | — | — |
| | Nitrate | | | | | |

Measured parameter: Assay (%);
Limits of measured parameter: 90-110
Method of measurement: HPLC Method

xvii) PRODUCT: Sodium Fusidate+Clobetasol Propionate+Terbinafine Hydrochloride Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Clobetasol Propionate | USP | 0.05% |
| iii) Terbinafine Hydrochloride | BP | 1.0% |

TABLE 51
Description Test, Batch No. SCT-01
Measured parameter: Physical appearance
Best value of measured parameter: Homogeneous White
to off White Viscous cream;
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to | White to | White to | White to |
| | off White | off White | off White | off White |
| | viscous | viscous | viscous | viscous |
| | cream | cream | cream | cream |
| 30° C. | — | Homogenous | Homogenous | Homogenous |
| 65% RH | | White to | White to | White to |
| | | off White | off White | off White |
| | | viscous | viscous | viscous |
| | | cream | cream | cream |
| 25° C. | — | Homogenous | Homogenous | Homogenous |
| 60% RH | | White to | White to | White to |
| | | off White | off White | off White |
| | | viscous | viscous | viscous |
| | | cream | cream | cream |
| Temperature | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 52
Assay (%) Test, Batch No. SCT-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% RH | i) Fusidic acid | 108.78 | 108.73 | 108.64 | 108.58 |
| | ii) Clobetasol | 108.51 | 108.44 | 108.32 | 108.25 |
| | Propionate | | | | |
| | iii) Terbinafine | 107.65 | 107.58 | 107.49 | 107.34 |
| | Hydrochloride | | | | |
| 30° C. 65% RH | i) Fusidic acid | — | 108.71 | 108.68 | 108.42 |
| | ii) Clobetasol | — | 108.48 | 108.36 | 108.32 |
| | Propionate | | | | |
| | iii) Terbinafine | — | 107.62 | 107.59 | 107.44 |
| | Hydrochloride | | | | |
| 25° C. 60% RH | i) Fusidic acid | — | 108.62 | 108.54 | 108.45 |
| | ii) Clobetasol | — | 108.44 | 108.36 | 108.28 |
| | Propionate | | | | |
| | iii) Terbinafine | — | 107.54 | 107.41 | 107.34 |
| | Hydrochloride | | | | |
| Temperature | i) Fusidic acid | — | 108.42 | — | — |
| cycling | ii) Clobetasol | — | 108.32 | — | — |
| | Propionate | | | | |
| | iii) Terbinafine | | 107.22 | | |
| | Hydrochloride | | | | |
| Freezthaw | i) Fusidic acid | — | 108.48 | — | — |
| | ii) Clobetasol | — | 108.21 | — | — |
| | Propionate | | | | |
| | iii) Terbinafine | — | 107.26 | — | — |
| | Hydrochloride | | | | |

TABLE 53
pH Test, Batch No. SCT-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.12 | 4.11 | 4.10 | 4.10 |
| 30° C. 65% RH | — | 4.12 | 4.11 | 4.11 |
| 25° C. 60% RH | — | 4.11 | 4.11 | 4.10 |
| Temperature | — | 4.12 | — | — |
| cycling | | | | |
| Freezthaw | — | 4.11 | — | — |

xviii) PRODUCT: Sodium Fusidate+Clobetasol Propionate+Ketoconazole Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Clobetasol Propionate | USP | 0.05% |
| iii) Ketoconazole | IP | 2.0% |

TABLE 54
Description Test, Batch No. SCK-01
Measured parameter: Physical appearance
Best value of measured parameter:
Homogeneous White to off White Viscous cream
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to | White to | White to | White to |
| | off White | off White | off White | off White |
| | viscous | viscous | viscous | viscous |
| | cream | cream | cream | cream |
| 30° C. | — | Homogenous | Homogenous | Homogenous |
| 65% RH | | White to | White to | White to |
| | | off White | off White | off White |
| | | viscous | viscous | viscous |
| | | cream | cream | cream |
| 25° C. | — | Homogenous | Homogenous | Homogenous |
| 60% RH | | White to | White to | White to |
| | | off White | off White | off White |
| | | viscous | viscous | viscous |
| | | cream | cream | cream |
| Temperature | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 55
pH Test, Batch No. SCK-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.55 | 4.54 | 4.53 | 4.53 |
| 30° C. 65% RH | — | 4.54 | 4.53 | 4.53 |
| 25° C. 60% RH | — | 4.53 | 4.52 | 4.52 |
| Temperature | — | 4.53 | — | — |
| cycling | | | | |
| Freezthaw | — | 4.52 | — | — |

TABLE 56
Assay (%) Test, Batch No. SCK-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 109.10 | 109.08 | 108.92 | 108.89 |
| RH | ii) Clobetasol | 108.41 | 108.34 | 108.22 | 108.15 |
| | Propionate | | | | |
| | iii) Ketoconazole | 107.85 | 107.78 | 107.69 | 107.54 |
| 30° C. 65% | i) Fusidic acid | — | 109.08 | 109.02 | 108.89 |
| RH | ii) Clobetasol | — | 108.40 | 108.38 | 108.28 |
| | Propionate | | | | |
| | iii) Ketoconazole | — | 107.75 | 107.69 | 107.54 |
| 25° C. 60% | i) Fusidic acid | — | 109.10 | 109.08 | 109.02 |
| RH | ii) Clobetasol | — | 108.38 | 108.34 | 108.31 |
| | Propionate | | | | |
| | iii) Ketoconazole | — | 107.75 | 107.64 | 107.54 |
| Temperature | i) Fusidic acid | — | 108.92 | — | — |
| cycling | ii) Clobetasol | — | 108.35 | — | — |
| | Propionate | | | | |
| | iii) Ketoconazole | | 107.42 | | |
| Freezthaw | i) Fusidic acid | — | 108.98 | — | — |
| | ii) Clobetasol | — | 108.31 | — | — |
| | Propionate | | | | |
| | iii) Ketoconazole | — | 107.56 | — | — |

xix) PRODUCT: Sodium Fusidate+Beclomethasone Dipropionate+Miconazole Nitrate Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Beclomethasone dipropionate | IP | 0.025% |
| iii) Miconazole Nitrate | IP | 2.0% |

TABLE 57
Description Test, Batch No. SBM-01
Measured parameter: Physical appearance
Best value of measured parameter:
Homogeneous White to off White Viscous cream;
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to off | White to | White to | White to |
| | White viscous | off White | off White | off White |
| | cream | viscous | viscous | viscous |
| | | cream | cream | cream |
| 30° C. | — | Homogenous | Homogenous | Homogenous |
| 65% RH | | White to | White to | White to |
| | | off White | off White | off White |
| | | viscous | viscous | viscous |
| | | cream | cream | cream |
| 25° C. | — | Homogenous | Homogenous | Homogenous |
| 60% RH | | White to | White to | White to |
| | | off White | off White | off White |
| | | viscous | viscous | viscous |
| | | cream | cream | cream |
| Temperature | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 58
pH Test, Batch No. SBM-01
Measured parameter: pH; Limits of measured parameter: 3-6
Method of measurement: Digital pH Meter

| | | 1st | 2nd | 3rd |
|---|---|---|---|---|
| Conditions | Initial | Month | Month | Month |
| 40° C. 75% RH | 4.42 | 4.42 | 4.41 | 4.42 |
| 30° C. 65% RH | — | 4.41 | 4.42 | 4.41 |
| 25° C. 60% RH | — | 4.42 | 4.41 | 4.42 |
| Temperature | — | 4.41 | — | — |
| cycling | | | | |
| Freezthaw | — | 4.41 | — | — |

TABLE 59
Assay (%) Test, Batch No. SBM-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 108.77 | 108.66 | 108.56 | 108.41 |
| RH | ii) Beclomethasone | 108.56 | 108.51 | 108.32 | 108.11 |
| | dipropionate | | | | |
| | iii) Miconazole | 107.65 | 107.54 | 107.44 | 107.32 |
| | Nitrate | | | | |
| 30° C. 65% | i) Fusidic acid | — | 108.73 | 108.61 | 108.46 |
| RH | ii) Beclomethasone | | 108.48 | 108.42 | 108.20 |
| | dipropionate | | | | |
| | iii) Miconazole | | 107.55 | 107.44 | 107.32 |
| | Nitrate | | | | |
| 25° C. 60% | i) Fusidic acid | — | 108.64 | 108.52 | 108.48 |
| RH | ii) Beclomethasone | | 108.54 | 108.42 | 108.21 |
| | dipropionate | | | | |
| | iii) Miconazole | | 107.48 | 107.40 | 107.38 |
| | Nitrate | | | | |
| Temperature | i) Fusidic acid | — | 107.83 | — | — |
| cycling | ii) Beclomethasone | | 108.51 | — | — |
| | dipropionate | | | | |
| | iii) Miconazole | | 107.25 | | |
| | Nitrate | | | | |
| Freezthaw | i) Fusidic acid | — | 108.41 | — | — |
| | ii) Beclomethasone | — | 108.15 | — | — |
| | dipropionate | | | | |
| | iii) Miconazole | | 107.14 | — | — |
| | Nitrate | | | | |

### xx) PRODUCT: Sodium Fusidate+Beclomethasone Dipropionate+Terbinafine Hydrochloride Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Beclomethasone dipropionate | IP | 0.025% |
| iii) Terbinafine Hydrochloride | BP | 1.0% |

TABLE 60
Description Test, Batch No. SLT-01
Measured parameter: Physical appearance
Best value of measured parameter: Homogeneous White to off White
Viscous cream; Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to off | White to | White to off | White to off |
| | White viscous | off White | White | White viscous |
| | cream | viscous | viscous | cream |
| | | cream | cream | |
| 30° C. | — | Homogenous | Homogenous | Homogenous |
| 65% RH | | White to | White to off | White to off |
| | | off White | White | White viscous |
| | | viscous | viscous | cream |
| | | cream | cream | |
| 25° C. | — | Homogenous | Homogenous | Homogenous |
| 60% RH | | White to | White to off | White to off |
| | | off White | White | White viscous |
| | | viscous | viscous | cream |
| | | cream | cream | |
| Temp | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 61
Assay (%) Test, Batch No. SLT-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 108.27 | 108.26 | 108.22 | 108.11 |
| RH | ii) Beclomethasone | 108.66 | 108.55 | 108.42 | 108.31 |
| | dipropionate | | | | |
| | iii) Terbinafine | 107.75 | 107.64 | 107.54 | 107.42 |
| | Hydrochloride | | | | |
| 30° C. 65% | i) Fusidic acid | — | 108.23 | 108.21 | 108.16 |
| RH | ii) Beclomethasone | | 108.58 | 108.44 | 108.32 |
| | dipropionate | | | | |
| | iii) Terbinafine | | 107.58 | 107.48 | 107.38 |
| | Hydrochloride | | | | |
| 25° C. 60% | i) Fusidic acid | — | 108.24 | 108.22 | 108.18 |
| RH | ii) Beclomethasone | | 108.64 | 108.54 | 108.31 |
| | dipropionate | | | | |
| | iii) Terbinafine | | 107.68 | 107.50 | 107.48 |
| | Hydrochloride | | | | |
| Temp | i) Fusidic acid | — | 108.13 | — | — |
| cycling | ii) Beclomethasone | | 108.25 | — | — |
| | dipropionate | | | | |
| | iii) Terbinafine | | 107.45 | | |
| | Hydrochloride | | | | |
| Freezthaw | i) Fusidic acid | — | 108.21 | — | — |
| | ii) Beclomethasone | — | 108.15 | — | — |
| | dipropionate | | | | |
| | iii) Terbinafine | | 107.34 | — | — |
| | Hydrochloride | | | | |

TABLE 62
pH Test, Batch No. SLT-01
Measured parameter: pH; Limits of measured parameter: 3-6
Method of measurement: Digital pH Meter

| | | 1st | 2nd | 3rd |
|---|---|---|---|---|
| Conditions | Initial | Month | Month | Month |
| 40° C. 75% RH | 4.12 | 4.11 | 4.10 | 4.09 |
| 30° C. 65% RH | — | 4.12 | 4.11 | 4.10 |
| 25° C. 60% RH | — | 4.11 | 4.10 | 4.10 |
| Temperature | — | 4.11 | — | — |
| cycling | | | | |
| Freezthaw | — | 4.10 | — | — |

xxi) PRODUCT: Sodium Fusidate+Beclomethasone Dipropionate+Ketoconazole Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Beclomethasone dipropionate | IP | 0.025% |
| iii) Ketoconazole | IP | 2.0% |

TABLE 63
Description Test, Batch No. SLK-01
Measured parameter: Physical appearance
Best value of measured parameter: Homogeneous White to off
White Viscous cream
Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to off | White to | White to | White to |
| | White viscous | off White | off White | off White |
| | cream | viscous | viscous | viscous |
| | | cream | cream | cream |
| 30° C. | — | Homogenous | Homogenous | Homogenous |
| 65% RH | | White to | White to | White to |
| | | off White | off White | off White |
| | | viscous | viscous | viscous |
| | | cream | cream | cream |
| 25° C. | — | Homogenous | Homogenous | Homogenous |
| 60% RH | | White to | White to | White to |
| | | off White | off White | off White |
| | | viscous | viscous | viscous |
| | | cream | cream | cream |
| Temperature | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 64
pH Test, Batch No. SLK-01
Measured parameter: pH; Limits of measured parameter: 3-6
Method of measurement: Digital pH Meter

| | | | 2nd | 3rd |
|---|---|---|---|---|
| Conditions | Initial | 1st Month | Month | Month |
| 40° C. 75% RH | 4.22 | 4.21 | 4.20 | 4.19 |
| 30° C. 65% RH | — | 4.22 | 4.21 | 4.20 |
| 25° C. 60% RH | — | 4.21 | 4.20 | 4.20 |
| Temperature | — | 4.21 | — | — |
| cycling | | | | |
| Freezthaw | — | 4.20 | — | — |

TABLE 65
Assay (%) Test, Batch No. SLK-01
Measured parameter: Assay (%)
Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 108.67 | 108.56 | 108.42 | 108.21 |
| RH | ii) Beclomethasone | 107.56 | 107.52 | 107.42 | 107.31 |
| | dipropionate | | | | |
| | iii) Ketoconazole | 107.85 | 107.74 | 107.64 | 107.52 |
| 30° C. 65% | i) Fusidic acid | — | 108.63 | 108.51 | 108.46 |
| RH | ii) Beclomethasone | | 107.58 | 107.44 | 107.32 |
| | dipropionate | | | | |
| | iii) Ketoconazole | | 107.75 | 107.64 | 107.48 |
| 25° C. 60% | i) Fusidic acid | — | 108.54 | 108.42 | 108.38 |
| RH | ii) Beclomethasone | | 107.64 | 107.54 | 107.31 |
| | dipropionate | | | | |
| | iii) Ketoconazole | | 107.81 | 107.70 | 107.58 |
| Temperature | i) Fusidic acid | — | 108.41 | — | — |
| cycling | ii) Beclomethasone | | 107.25 | — | — |
| | dipropionate | | | | |
| | iii) Ketoconazole | | 107.55 | | |
| Freezthaw | i) Fusidic acid | — | 108.51 | — | — |
| | ii) Beclomethasone | — | 107.15 | — | — |
| | dipropionate | | | | |
| | iii) Ketoconazole | | 107.44 | — | — |

xxii) PRODUCT: Sodium Fusidate+Betamethasone Dipropionate+Miconazole Nitrate Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Betamethasone dipropionate | USP | 0.05% |
| iii) Miconazole Nitrate | IP | 2.0% |

TABLE 66
Description Test, Batch No. SMB-01
Measured parameter: Physical appearance
Best value of measured parameter: Homogeneous White to off White
Viscous cream; Method of measurement: Observation by naked eye

| Conditions | Initial | 1st Month | 2nd Month | 3rd Month |
|---|---|---|---|---|
| 40° C. 75% | Homogenous | Homogenous | Homogenous | Homogenous |
| RH | White to | White to | White to | White to |
| | off White | off White | off White | off White |
| | viscous | viscous | viscous | viscous |
| | cream | cream | cream | cream |
| 30° C. 65% | — | Homogenous | Homogenous | Homogenous |
| RH | | White to | White to | White to |
| | | off White | off White | off White |
| | | viscous | viscous | viscous |
| | | cream | cream | cream |
| 25° C. 60% | — | Homogenous | Homogenous | Homogenous |
| RH | | White to | White to | White to |
| | | off White | off White | off White |
| | | viscous | viscous | viscous |
| | | cream | cream | cream |
| Temperature | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 67
pH Test, Batch No. SMB-01
Measured parameter: pH
Limits of measured parameter: 3-6
Method of measurement: Digital pH Meter

| Conditions | Initial | 1st Month | 2nd Month | 3rd Month |
|---|---|---|---|---|
| 40° C. 75% RH | 4.41 | 4.40 | 4.41 | 4.40 |
| 30° C. 65% RH | — | 4.41 | 4.40 | 4.40 |
| 25° C. 60% RH | — | 4.39 | 4.41 | 4.40 |
| Temperature | — | 4.39 | — | — |
| cycling | | | | |
| Freezthaw | — | 4.38 | — | — |

TABLE 68
Assay (%) Test, Batch No. SMB-01
Measured parameter: Assay (%)
Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. | i) Fusidic acid | 108.72 | 108.68 | 108.50 | 108.42 |
| 75% RH | ii) Betamethasone | 107.42 | 107.34 | 107.28 | 107.18 |
| | dipropionate | | | | |
| | iii) Miconazole Nitrate | 107.95 | 107.85 | 107.75 | 107.55 |
| 30° C. | i) Fusidic acid | — | 108.70 | 108.64 | 108.58 |
| 65% RH | ii) Betamethasone | — | 107.40 | 107.38 | 107.32 |
| | dipropionate | | | | |
| | iii) Miconazole Nitrate | — | 107.85 | 107.75 | 107.56 |
| 25° C. | i) Fusidic acid | — | 108.65 | 108.55 | 108.41 |
| 60% RH | ii) Betamethasone | — | 107.35 | 107.28 | 107.21 |
| | dipropionate | | | | |
| | iii) Miconazole Nitrate | — | 107.86 | 107.58 | 107.29 |
| Temper- | i) Fusidic acid | — | 108.53 | — | — |
| ature | ii) Betamethasone | — | 107.18 | — | — |
| cycling | dipropionate | | | | |
| | iii) Miconazole Nitrate | — | 107.26 | | |
| Freezthaw | i) Fusidic acid | — | 108.61 | — | — |
| | ii) Betamethasone | — | 107.38 | — | — |
| | dipropionate | | | | |
| | iii) Miconazole Nitrate | — | 107.68 | — | — |

### Xxiii) PRODUCT: Sodium Fusidate+Betamethasone Dipropionate+Terbinafine Hydrochloride Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Betamethasone dipropionate | USP | 0.05% |
| iii) Terbinafine Hydrochloride | BP | 1.0% |

TABLE 69
Description Test, Batch No. STB-01
Measured parameter: Physical appearance
Best value of measured parameter: Homogeneous White to off White
Viscous cream; Method of measurement: Observation by naked eye

| Conditions | Initial | 1st Month | 2nd Month | 3rd Month |
|---|---|---|---|---|
| 40° C. 75% | Homogenous | Homogenous | Homogenous | Homogenous |
| RH | White to off | White to off | White to off | White to off |
| | White | White | White | White |
| | viscous | viscous | viscous | viscous |
| | cream | cream | cream | cream |
| 30° C. 65% | — | Homogenous | Homogenous | Homogenous |
| RH | | White to off | White to off | White to off |
| | | White | White | White |
| | | viscous | viscous | viscous |
| | | cream | cream | cream |
| 25° C. 60% | — | Homogenous | Homogenous | Homogenous |
| RH | | White to off | White to off | White to off |
| | | White | White | White |
| | | viscous | viscous | viscous |
| | | cream | cream | cream |
| Temp cycling | — | Homogenous | — | — |
| | | White to off | | |
| | | White | | |
| | | viscous | | |
| | | cream | | |
| Freezthaw | — | Homogenous | — | — |
| | | White to off | | |
| | | White | | |
| | | viscous | | |
| | | cream | | |

TABLE 70
Assay (%) Test, Batch No. STB-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 107.72 | 107.68 | 107.50 | 107.42 |
| RH | ii) Betamethasone | 107.52 | 107.44 | 107.38 | 107.28 |
| | dipropionate | | | | |
| | iii) Terbinafine | 107.85 | 107.75 | 107.65 | 107.45 |
| | Hydrochloride | | | | |
| 30° C. 65% | i) Fusidic acid | — | 107.70 | 107.67 | 107.58 |
| RH | ii) Betamethasone | — | 107.41 | 107.28 | 107.22 |
| | dipropionate | | | | |
| | iii) Terbinafine | — | 107.75 | 107.65 | 107.52 |
| | Hydrochloride | | | | |
| 25° C. 60% | i) Fusidic acid | — | 107.65 | 107.55 | 107.41 |
| RH | ii) Betamethasone | — | 107.45 | 107.38 | 107.31 |
| | dipropionate | | | | |
| | iii) Terbinafine | — | 107.76 | 107.63 | 107.39 |
| | Hydrochloride | | | | |
| Temperature | i) Fusidic acid | — | 107.53 | — | — |
| cycling | ii) Betamethasone | — | 107.28 | — | — |
| | dipropionate | | | | |
| | iii) Terbinafine | — | 107.36 | | |
| | Hydrochloride | | | | |
| Freezthaw | i) Fusidic acid | — | 107.61 | — | — |
| | ii) Betamethasone | — | 107.03 | — | — |
| | dipropionate | | | | |
| | iii) Terbinafine | — | 107.58 | — | — |
| | Hydrochloride | | | | |

TABLE 71
pH Test, Batch No. STB-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.31 | 4.30 | 4.31 | 4.30 |
| 30° C. 65% RH | — | 4.31 | 4.30 | 4.30 |
| 25° C. 60% RH | — | 4.30 | 4.29 | 4.30 |
| Temperature | — | 4.31 | — | — |
| cycling | | | | |
| Freezthaw | — | 4.30 | — | — |

xxiv) PRODUCT: Sodium Fusidate+Betamethasone Dipropionate+Ketoconazole Cream

### PACK: Aluminum Collapsible Tube

| Composition: | Each gm contains: | |
|---|---|---|
| i) Sodium Fusidate BP Equivalent to Fusidic Acid | BP | 2.0% |
| ii) Betamethasone dipropionate | USP | 0.05% |
| iii) Ketoconazole | IP | 2.0% |

TABLE 72
Description Test, Batch No. SSK-01
Measured parameter: Physical appearance
Best value of measured parameter: Homogeneous White to off White
Viscous cream; Method of measurement: Observation by naked eye

| Conditions | Initial | 1st Month | 2nd Month | 3rd Month |
|---|---|---|---|---|
| 40° C. | Homogenous | Homogenous | Homogenous | Homogenous |
| 75% RH | White to | White to | White to | White to |
| | off White | off White | off White | off White |
| | viscous | viscous | viscous | viscous |
| | cream | cream | cream | cream |
| 30° C. | — | Homogenous | Homogenous | Homogenous |
| 65% RH | | White to | White to | White to |
| | | off White | off White | off White |
| | | viscous | viscous | viscous |
| | | cream | cream | cream |
| 25° C. | — | Homogenous | Homogenous | Homogenous |
| 60% RH | | White to | White to | White to |
| | | off White | off White | off White |
| | | viscous | viscous | viscous |
| | | cream | cream | cream |
| Temperature | — | Homogenous | — | — |
| cycling | | White to | | |
| | | off White | | |
| | | viscous | | |
| | | cream | | |
| Freezthaw | — | Homogenous | — | — |
| | | White to | | |
| | | off White | | |
| | | viscous | | |
| | | cream | | |

TABLE 73
pH Test, Batch No. SSK-01
Measured parameter: pH; Limits of measured parameter: 3-6
Method of measurement: Digital pH Meter

| Conditions | Initial | 1st Month | 2nd Month | 3rd Month |
|---|---|---|---|---|
| 40° C. 75% RH | 4.28 | 4.27 | 4.28 | 4.27 |
| 30° C. 65% RH | — | 4.28 | 4.27 | 4.26 |
| 25° C. 60% RH | — | 4.27 | 4.27 | 4.26 |
| Temperature | — | 4.26 | — | — |
| cycling | | | | |
| Freezthaw | — | 4.27 | — | — |

TABLE 74
Assay (%) Test, Batch No. SSK-01
Measured parameter: Assay (%); Limits of measured parameter: 90-110
Method of measurement: HPLC Method

| | | | 1st | 2nd | 3rd |
|---|---|---|---|---|---|
| Conditions | Assay (%) | Initial | Month | Month | Month |
| 40° C. 75% | i) Fusidic acid | 108.72 | 108.68 | 108.50 | 108.42 |
| RH | ii) Betamethasone | 108.55 | 108.43 | 108.37 | 108.24 |
| | dipropionate | | | | |
| | iii) Ketoconazole | 107.75 | 107.65 | 107.45 | 107.35 |
| 30° C. 65% | i) Fusidic acid | — | 108.70 | 108.67 | 108.58 |
| RH | ii) Betamethasone | — | 108.42 | 108.28 | 107.21 |
| | dipropionate | | | | |
| | iii) Ketoconazole | — | 107.65 | 107.55 | 107.42 |
| 25° C. 60% | i) Fusidic acid | — | 108.65 | 108.55 | 108.41 |
| RH | ii) Betamethasone | — | 108.45 | 108.38 | 108.31 |
| | dipropionate | | | | |
| | iii) Ketoconazole | — | 107.66 | 107.53 | 107.49 |
| Temperature | i) Fusidic acid | — | 108.53 | — | — |
| cycling | ii) Betamethasone | — | 108.28 | — | — |
| | dipropionate | | | | |
| | iii) Ketoconazole | — | 107.16 | | |
| Freezthaw | i) Fusidic acid | — | 108.61 | — | — |
| | ii) Betamethasone | — | 108.13 | — | — |
| | dipropionate | | | | |
| | iii) Ketoconazole | — | 107.65 | — | — |

From the above data, it is evident that product of the present invention is quite stable at ambient conditions and also at elevated temperature & humid conditions of storage.

According to the preferred embodiment of the present invention, there is provided a single dose composition comprising at least one steroid, at least one antifungal and at least one antibacterial agent for the topical treatment of bacterial/fungal skin infections and inflammations on human skin, the composition comprising a steroid selected from a group comprising Betamethasone Valerate, Fluticasone Propionate, Mometasone Furoate, Dexamethasone Acetate, Hydrocortisone Acetate, Clobetasol Propionate, Beclomethasone Dipropionate, Betamethasone Dipropionate and the like, and an antifungal selected from a group comprising Miconazole Nitrate, Terbinafine Hydrochloride, Ketoconazole and Fusidic acid made in situ by a conversion of Sodium Fusidate, a cream base containing primary and secondary emulsifiers, waxy materials, co-solvents, and acids, and water.

The proportions of various components of the preferred embodiment are as follows:
a. Fusidic acid from about 0.1% (w/w) to about 25% (w/w), preferably from about 0.5% (w/w) to about 5% (w/w) and more preferably about 2.00% (w/w), which has been converted in situ from Sodium Fusidate from about 0.1% (w/w) to about 25% (w/w), preferably from about 0.5% (w/w) to about 5% (w/w) and more preferably about 2.08% (w/w), and from about 0.001% (w/w) to about 5% (w/w), preferably from about 0.005% (w/w) to about 2.00% (w/w), and most preferably from about 0.05% (w/w) to 1.0% (w/w), of a corticosteroid active compound, and from about 0.01% (w/w) to about 10% (w/w), preferably from about 0.1% (w/w) to about 5.00% (w/w), and most preferably from about 1.0% (w/w) to 2.0% (w/w), of an antifungal active compound,
b. a cream base containing primary and secondary emulsifiers, waxy materials, co-solvents, acids, and water wherein
   primary and secondary emulsifiers are selected from a group comprising Cetostearyl alcohol, Cetomacrogol-1000, Polysorbate-80, Span-80 and the like from about 1% (w/w) to 15% (w/w), preferably 15% (w/w), more preferably 14.5% (w/w)
   waxy materials are selected from a group comprising White Soft Paraffin, Liquid Paraffin, Hard Paraffin and the like from about 5% (w/w) to 20% (w/w), preferably 15% (w/w), more preferably 12.5% (w/w),
   co-solvents are selected from a group comprising Propylene Glycol, Hexylene Glycol, PolyEthylene Glycol-400 and the like from about 5% (w/w) to 40% (w/w), preferably 30% (w/w), more preferably 25% (w/w),
   acids are selected from a group comprising HCl, H2So4, HNO3, Lactic acid and the like from about 0.005% (w/w) to 0.5% (w/w), preferably 0.3% (w/w), more preferably 0.25% (w/w), and
   water in the amount in the range of 20% (w/w) to 75% (w/w), preferably 35% (w/w) to 50% (w/w), more preferably 38% (w/w) to 43% (w/w), preferably purified water.

In another embodiment of the present invention the product of the preferred embodiment is further provided with preservatives, wherein said preservatives are selected from a group comprising Methylparaben, Propylparaben, Chlorocresol, Potassium sorbate, Benzoic acid and the like from about 0.05% (w/w) to 0.5% (w/w), preferably 0.3% (w/w), more preferably 0.2% (w/w).

In a still further embodiment of the present invention, the product of the preferred embodiment is further provided with a buffering agent selected from a group comprising Di Sodium Hydrogen Ortho Phosphate, Sodium Hydrogen Ortho Phosphate and the like from about 0.01% (w/w) to 1.00% (w/w), preferably 0.5% (w/w), more preferably 0.05% (w/w).

In yet another embodiment of the present invention, the product of the preferred embodiment is further provided with an anti oxidants are selected from a group comprising Butylated Hydroxy Anisole, Butylated Hydroxy Toluene and the like from about 0.001% (w/w) to 5% (w/w), preferably 0.1% (w/w), more preferably 0.01% (w/w).

In a further embodiment of the present invention, the product of the preferred embodiment is further provided with a chelating selected from a group comprising Disodium EDTA and the like from about 0.01% (w/w) to 1% (w/w), preferably 0.5% (w/w), more preferably 0.1% (w/w).

In still another embodiment of the present invention, the product of the preferred embodiment is further provided with a humectant selected from a group comprising Glycerin, Sorbitol, Propylene glycol and the like from about 5% (w/w) to 40% (w/w) preferably 30% (w/w), more preferably 25% (w/w).

In another embodiment of the present invention, the product of the preferred embodiment further is provided with at least one component selected from a group comprising buffering agents, preservatives, anti oxidants, chelating agents, humectants, or any combination thereof in respective proportions disclosed in the earlier described embodiments.

In a further embodiment of the present invention, a novel dermaceutical cream is disclosed wherein sodium fusidate is converted in-situ under totally oxygen free environment by slow addition of an acid, into Fusidic acid of a molecular dispersion form (due to the presence of a co-solvent) at the intermediate stage, and which Fusidic acid regenerates into an extremely finely dispersed form when added to a final cream base, thereby resulting in a finely and homogeneously dispersed Fusidic acid in the final cream; all operations of converting sodium fusidate into Fusidic acid carried out preferably in an environment free of atmospheric oxygen.

Composition of the various samples used for obtaining the foregoing experimental results are now provided below. These compositions also represent some of the various embodiments of the present invention.

TABLE 75
Sodium Fusidate + Betamethasone Valerate + Miconazole Nitrate Cream

| S. | | | % |
|---|---|---|---|
| No | Ingredients | Specification | (w/w) |
| 1 | Fusidic acid made from Sodium Fusidate | BP | 2.00 |
| 2 | Betamethasone Valerate | IP | 0.12 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate anhydrous | IP | 0.05 |
| 13 | Purified Water | IP | 39.5 |

TABLE 76
Sodium Fusidate + Betamethasone Valerate + Terbinafine
Hydrochloride Cream

| S. | | | % |
|---|---|---|---|
| No | Ingredients | Specification | (w/w) |
| 1 | Fusidic acid made from Sodium Fusidate | BP | 2.00 |
| 2 | Betamethasone Valerate | IP | 0.12 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate anhydrous | IP | 0.05 |
| 13 | Purified Water | IP | 40.5 |

TABLE 77
Sodium Fusidate + Betamethasone Valerate + Ketoconazole Cream

| S. | | | % |
|---|---|---|---|
| No | Ingredients | Specification | (w/w) |
| 1 | Fusidic acid made from Sodium Fusidate | BP | 2.00 |
| 2 | Betamethasone Valerate | IP | 0.12 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate anhydrous | IP | 0.05 |
| 13 | Purified Water | IP | 39.5 |

TABLE 78
Sodium Fusidate + Fluticasone Propionate + Miconazole Nitrate Cream

| S. | | | % |
|---|---|---|---|
| No | Ingredients | Specification | (w/w) |
| 1 | Fusidic acid made from Sodium Fusidate | BP | 2.00 |
| 2 | Fluticasone Propionate | BP | 0.05 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate anhydrous | IP | 0.05 |
| 13 | Purified Water | IP | 39.5 |

TABLE 79
Sodium Fusidate + Fluticasone Propionate + Terbinafine
Hydrochloride Cream

| | | | % |
|---|---|---|---|
| S. No | Ingredients | Specification | (w/w) |
| 1 | Fusidic acid made from Sodium Fusidate | BP | 2.00 |
| 2 | Fluticasone Propionate | BP | 0.05 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 40.5 |

TABLE 80
Sodium Fusidate + Fluticasone Propionate + Ketoconazole Cream

| | | | % |
|---|---|---|---|
| S. No | Ingredients | Specification | (w/w) |
| 1 | Fusidic acid made from Sodium Fusidate | BP | 2.00 |
| 2 | Fluticasone Propionate | BP | 0.05 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.5 |

TABLE 81
Sodium Fusidate + Mometasone Furoate + Miconazole Nitrate Cream

| S. | | | % |
|---|---|---|---|
| No | Ingredients | Specification | (w/w) |
| 1 | Fusidic acid made from Sodium Fusidate | BP | 2.00 |
| 2 | Mometasone Furoate | USP | 0.1 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate anhydrous | IP | 0.05 |
| 13 | Purified Water | IP | 39.5 |

TABLE 82
Sodium Fusidate + Mometasone Furoate + Terbinafine
Hydrochloride Cream

| | | | % |
|---|---|---|---|
| S. No | Ingredients | Specification | (w/w) |
| 1 | Fusidic acid made from Sodium Fusidate | BP | 2.00 |
| 2 | Mometasone Furoate | USP | 0.1 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 40.5 |

TABLE 83
Sodium Fusidate + Mometasone Furoate + Ketoconazole Cream

| | | | % |
|---|---|---|---|
| S. No | Ingredients | Specification | (w/w) |
| 1 | Fusidic acid made from Sodium Fusidate | BP | 2.00 |
| 2 | Mometasone Furoate | USP | 0.1 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.5 |

TABLE 84
Sodium Fusidate + Dexamethasone Acetate + Miconazole Nitrate Cream

| S. | | | % |
|---|---|---|---|
| No | Ingredients | Specification | (w/w) |
| 1 | Fusidic acid made from Sodium Fusidate | BP | 2.00 |
| 2 | Dexamethasone Acetate | BP | 0.1 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate anhydrous | IP | 0.05 |
| 13 | Purified Water | IP | 39.5 |

TABLE 85
Sodium Fusidate + Dexamethasone Acetate + Terbinafine
Hydrochloride Cream

| | | | % |
|---|---|---|---|
| S. No | Ingredients | Specification | (w/w) |
| 1 | Fusidic acid made from Sodium Fusidate | BP | 2.00 |
| 2 | Dexamethasone Acetate | BP | 0.1 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 40.5 |

TABLE 86
Sodium Fusidate + Dexamethasone Acetate + Ketoconazole Cream

| | | | % |
|---|---|---|---|
| S. No | Ingredients | Specification | (w/w) |
| 1 | Fusidic acid made from Sodium Fusidate | BP | 2.00 |
| 2 | Dexamethasone Acetate | BP | 0.1 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.5 |

TABLE 87
Sodium Fusidate + Hydrocortisone Acetate + Miconazole Nitrate Cream

| | | | % |
|---|---|---|---|
| S. No | Ingredients | Specification | (w/w) |
| 1 | Fusidic acid made from Sodium Fusidate | BP | 2.00 |
| 2 | Hydrocortisone Acetate | IP | 1.00 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39 |

TABLE 88
Sodium Fusidate + Hydrocortisone Acetate + Terbinafine
Hydrochloride Cream

| | | | % |
|---|---|---|---|
| S. No | Ingredients | Specification | (w/w) |
| 1 | Fusidic acid made from Sodium Fusidate | BP | 2.00 |
| 2 | Hydrocortisone Acetate | IP | 1.00 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 40 |

TABLE 89
Sodium Fusidate + Hydrocortisone Acetate + Ketoconazole Cream

| | | | % |
|---|---|---|---|
| S. No | Ingredients | Specification | (w/w) |
| 1 | Fusidic acid made from Sodium Fusidate | BP | 2.00 |
| 2 | Hydrocortisone Acetate | IP | 1.00 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39 |

TABLE 90
Sodium Fusidate + Clobetasol Propionate + Miconazole Nitrate Cream

| | | | % |
|---|---|---|---|
| S. No | Ingredients | Specification | (w/w) |
| 1 | Fusidic acid made from Sodium Fusidate | BP | 2.00 |
| 2 | Clobetasol Propionate | USP | 0.05 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.5 |

TABLE 91
Sodium Fusidate + Clobetasol Propionate + Terbinafine
Hydrochloride Cream

| | | | % |
|---|---|---|---|
| S. No | Ingredients | Specification | (w/w) |
| 1 | Fusidic acid made from Sodium Fusidate | BP | 2.00 |
| 2 | Clobetasol Propionate | USP | 0.05 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 40.5 |

TABLE 92
Sodium Fusidate + Clobetasol Propionate + Ketoconazole Cream

| | | | % |
|---|---|---|---|
| S. No | Ingredients | Specification | (w/w) |
| 1 | Fusidic acid made from Sodium Fusidate | BP | 2.00 |
| 2 | Clobetasol Propionate | USP | 0.05 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.5 |

TABLE 93
Sodium Fusidate + Beclomethasone Dipropionate + Miconazole
Nitrate Cream

| | | | % |
|---|---|---|---|
| S. No | Ingredients | Specification | (w/w) |
| 1 | Fusidic acid made from Sodium Fusidate | BP | 2.00 |
| 2 | Beclomethasone Dipropionate | IP | 0.025 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.6 |

TABLE 94
Sodium Fusidate + Beclomethasone Dipropionate + Terbinafine
Hydrochloride Cream

| | | | % |
|---|---|---|---|
| S. No | Ingredients | Specification | (w/w) |
| 1 | Fusidic acid made from Sodium Fusidate | BP | 2.00 |
| 2 | Beclomethasone Dipropionate | IP | 0.025 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 40.6 |

TABLE 95
Sodium Fusidate + Beclomethasone Dipropionate + Ketoconazole Cream

| | | | % |
|---|---|---|---|
| S. No | Ingredients | Specification | (w/w) |
| 1 | Fusidic acid made from Sodium Fusidate | BP | 2.00 |
| 2 | Beclomethasone Dipropionate | IP | 0.025 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.6 |

TABLE 96
Sodium Fusidate + Betamethasone Dipropionate + Miconazole
Nitrate Cream

| | | | % |
|---|---|---|---|
| S. No | Ingredients | Specification | (w/w) |
| 1 | Fusidic acid made from Sodium Fusidate | BP | 2.00 |
| 2 | Betamethasone Dipropionate | USP | 0.05 |
| 3 | Miconazole Nitrate | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.6 |

TABLE 97
Sodium Fusidate + Betamethasone Dipropionate + Terbinafine
Hydrochloride Cream

| | | | % |
|---|---|---|---|
| S. No | Ingredients | Specification | (w/w) |
| 1 | Fusidic acid made from Sodium Fusidate | BP | 2.00 |
| 2 | Betamethasone Dipropionate | USP | 0.05 |
| 3 | Terbinafine Hydrochloride | BP | 1.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 40.6 |

TABLE 98
Sodium Fusidate + Betamethasone Dipropionate + Ketoconazole Cream

| | | | % |
|---|---|---|---|
| S. No | Ingredients | Specification | (w/w) |
| 1 | Fusidic acid made from Sodium Fusidate | BP | 2.00 |
| 2 | Betamethasone Dipropionate | USP | 0.05 |
| 3 | Ketoconazole | IP | 2.00 |
| 4 | Cetostearyl Alcohol | IP | 12.5 |
| 5 | White Soft Paraffin | IP | 12.5 |
| 6 | Polysorbate 80 | IP | 2 |
| 7 | Propylene Glycol | IP | 25 |
| 8 | Benzoic Acid | IP | 0.2 |
| 9 | Butylated Hydroxy Toluene | IP | 0.01 |
| 10 | Disodium Edetate | IP | 0.1 |
| 11 | 1M Nitric Acid | IP | 4.0 |
| 12 | Disodium hydrogen Orthophosphate | IP | 0.05 |
| | anhydrous | | |
| 13 | Purified Water | IP | 39.6 |

It is evident from the foregoing description that the present invention comprises the following embodiments.
1. A novel dermaceutical cream containing at least one corticosteroid, at least one antifungal and Fusidic acid which is made in situ under oxygen-free environment using Sodium Fusidate, wherein said cream comprises Fusidic acid made in situ by a conversion of Sodium Fusidate, and a cream base containing at least one of each of a preservative, a primary and secondary emulsifier, a waxy material, a co-solvents, an acid, and water, preferably purified water.
2. A novel dermaceutical cream as described in item 1, wherein said corticosteroid is added from about 0.001% (w/w) to about 5% (w/w), preferably from about 0.005% (w/w) to about 2.00% (w/w), and most preferably from about 0.05% (w/w) to 1.0% (w/w), and
said antifungal is added from about 0.01% (w/w) to about 10% (w/w), preferably from about 0.1% (w/w) to about 5.00% (w/w), and most preferably from about 1% (w/w) to 2.0% (w/w) and said Fusidic acid is present in an amount from about 0.1% (w/w) to about 25% (w/w), preferably from about 0.5% (w/w) to about 5% (w/w), and more preferably about 2.00% (w/w), and in which the amount of said Sodium Fusidate used to form in situ said Fusidic acid is in the range between about 0.1% (w/w) to about 25% (w/w), preferably from about 0.5% (w/w) to about 5% (w/w) and more preferably about 2.08% (w/w), and
   said preservatives is selected from a group comprising Methylparaben, Propylparaben, Chlorocresol, Potassium sorbate, Benzoic acid and the like, either singly or any combination thereof, to form a proportion from about 0.05% (w/w) to 0.5% (w/w), preferably 0.3% (w/w), more preferably 0.2% (w/w),
   said primary and secondary emulsifier is selected from a group comprising Cetostearyl alcohol, Cetomacrogol-1000, Polysorbate-80, Span-80 and the like, either singly or any combination thereof, to form a proportion from about 1% (w/w) to 15% (w/w), preferably 15% (w/w), more preferably 14.5% (w/w),
   said waxy material is selected from a group comprising White soft paraffin, Liquid Paraffin, Hard paraffin and the like, either singly or any combination thereof, to form a proportion from about 5% (w/w) to 20% (w/w), preferably 15% (w/w), more preferably 12.5% (w/w),
   said co-solvent is selected from a group comprising Propylene Glycol, Hexylene Glycol, PolyEthylene Glycol-400 and the like, either singly or any combination thereof, to form a proportion from about 5% (w/w) to 40% (w/w), preferably 30% (w/w), more preferably 25% (w/w),
   said acid is selected from a group comprising acids such as HCl, H2So4, HNO3, Lactic acid and the like, either singly or any combination thereof, to form a proportion from about 0.005% (w/w) to 0.5% (w/w), preferably 0.3% (w/w), more preferably 0.25% (w/w), and
   water in the amount in the range of 20% (w/w) to 75% (w/w), preferably 35% (w/w) to 50% (w/w), more preferably 38% (w/w) to 43% (w/w), preferably purified water.
3. A novel dermaceutical cream as described in item 1 which further comprises a buffering agent, wherein said buffering agent is selected from a group comprising Di Sodium Hydrogen Ortho Phosphate, Sodium Hydrogen Ortho Phosphate and the like, either singly or any combination thereof, to form a proportion from about 0.01% (w/w) to 1.00% (w/w), preferably 0.5% (w/w), more preferably 0.05% (w/w).

4. A novel dermaceutical cream as described in items 1 to 3 which further comprises an anti-oxidant, wherein said anti-oxidant is selected from a group comprising Butylated Hydroxy Anisole, Butylated Hydroxy Toluene and the like, either singly or any combination thereof, to form a proportion from about 0.001% (w/w) to 5% (w/w), preferably 0.1% (w/w), more preferably 0.01% (w/w).
5. A novel dermaceutical cream as described in items 1 to 4 which further comprises a chelating agent, wherein said chelating agent is selected from a group comprising Disodium EDTA and the like, either singly or any combination thereof, to form a proportion from about 0.01% (w/w) to 1% (w/w), preferably 0.5% (w/w), more preferably 0.1% (w/w).
6. A novel dermaceutical cream as described in items 1 to 5 which further comprises a humectant, wherein said humectant is selected from a group comprising Glycerin, Sorbitol, Propylene glycol and the like, either singly or any combination thereof, to form a proportion from about 5% (w/w) to 40% (w/w), preferably 30% (w/w), more preferably 25% (w/w).
7. A novel dermaceutical cream as described in items t to 6 wherein sodium fusidate is converted in-situ under totally oxygen free environment by slow addition of an acid, into Fusidic acid of a molecular dispersion form (due to the presence of a co-solvent) at the intermediate stage, and which Fusidic acid regenerates into an extremely finely dispersed form when added to a final cream base, thereby resulting in a finely and homogeneously dispersed Fusidic acid in the final cream; all operations of converting sodium fusidate into Fusidic acid carried out preferably in an environment free of atmospheric oxygen.
8. A novel dermaceutical cream as described in items 1 to 7 wherein said conversion of Sodium Fusidate into said Fusidic acid and the following formation of said Fusidic acid in a finely dispersed form in the final cream base take place in an oxygen-free environment.
9. A novel dermaceutical cream as described in item 8 wherein said oxygen-free environment comprises a gaseous environment formed of inert gas selected from a group comprising carbon dioxide, nitrogen, helium and the like.
10. A method of treating primary & secondary bacterial skin infections, fungal skin infections and inflammations said method comprising applying of a cream containing at least one corticosteroid, at least one antifungal and Fusidic acid which is made in situ under oxygen-free environment using Sodium Fusidate, wherein said cream comprises Fusidic acid made using Sodium Fusidate, a cream base containing a preservative, primary and secondary emulsifiers, waxy materials, co-solvents, acids, and water.
11. A method of treating primary & secondary bacterial skin infections, fungal skin infections and inflammations said method comprising applying of a cream as described in item 10, wherein said cream further comprises any of a group comprising a buffering agent, an anti oxidant, a chelating agent, and a humectant, or any combination thereof.
13. A method of treating primary & secondary bacterial skin infections, fungal skin infections and inflammations said method comprising applying of a cream as described in item 12, wherein said corticosteroid is added from about 0.001% (w/w) to about 5% (w/w), preferably from about 0.005% (w/w) to about 2.00% (w/w), and most preferably from about 0.05% (w/w) to 1.0% (w/w), and
said antifungal is added from about 0.01% (w/w) to about 10% (w/w), preferably from about 0.1% (w/w) to about 5.00% (w/w), and most preferably from about 1% (w/w) to 2.0% (w/w) and
said Fusidic acid is present in an amount from about 0.1% (w/w) to about 25% (w/w), preferably from about 0.5% (w/w) to about 5% (w/w), and more preferably about 2.00% (w/w), and in which the amount of Sodium Fusidate used to form in situ said Fusidic acid is in the range between about 0.1% (w/w) to about 25% (w/w), preferably from about 0.5% (w/w) to about 5% (w/w) and most preferably about 2.08% (w/w),
said primary and secondary emulsifier is selected from a group comprising Cetostearyl alcohol, Cetomacrogol-1000, Polysorbate-80, Span-80 and the like, either singly or any combination thereof, to form a proportion from about 1% (w/w) to 15% (w/w), preferably 15% (w/w), more preferably 14.5% (w/w),
said waxy material is selected from a group comprising white soft paraffin, liquid paraffin, Hard paraffin and the like, either singly or any combination thereof, to form a proportion from about 5% (w/w) to 20% (w/w), preferably 15% (w/w), more preferably 12.5% (w/w),
said co-solvent is selected from a group comprising Propylene Glycol, Hexylene Glycol, PolyEthylene Glycol-400 and the like, either singly or any combination thereof, to form a proportion from about 5% (w/w) to 40% (w/w), preferably 30% (w/w), more preferably 25% (w/w),
said acid is selected from a group comprising HCl, H2So4, HNO3, Lactic acid and the like, either singly or any combination thereof, to form a proportion from about 0.005% (w/w) to 0.5% (w/w), preferably 0.3% (w/w), more preferably 0.25% (w/w),
said preservative is selected from a group comprising Methylparaben, Propylparaben, Chlorocresol, Potassium sorbate, Benzoic acid and the like, either singly or any combination thereof, to form a proportion from about 0.05% (w/w) to 0.5% (w/w), preferably 0.3% (w/w), more preferably 0.2% (w/w),
said buffering agent is selected from a group comprising Di Sodium Hydrogen Ortho Phosphate, Sodium Hydrogen Ortho Phosphate and the like, either singly or any combination thereof, to form a proportion from about 0.01% (w/w) to 1.00% (w/w), preferably 0.5% (w/w), more preferably 0.05% (w/w),
said anti-oxidant is selected from a group comprising Butylated Hydroxy Anisole, Butylated Hydroxy Toluene and the like, either singly or any combination thereof, to form a proportion from about 0.001% (w/w) to 5% (w/w), preferably 0.1% (w/w), more preferably 0.01% (w/w),
said chelating agent is selected from a group comprising Disodium EDTA and the like, either singly or any combination thereof, to form a proportion from about 0.01% (w/w) to 1% (w/w), preferably 0.5% (w/w), more preferably 0.1% (w/w), and
said humectant is selected from a group comprising Glycerin, Sorbitol, Propylene glycol and the like, either singly or any combination thereof, to form a proportion from about 5% (w/w) to 40% (w/w), preferably 30% (w/w), more preferably 25% (w/w), and
said water in the amount in the range of 20% (w/w) to 75% (w/w), preferably 35% (w/w) to 50% (w/w), more preferably 38% (w/w) to 43% (w/w), preferably purified water.

It is evident from the foregoing description that the present invention has the following distinctions and advantages over the commercially available comparable products:
It has been prepared using Sodium Fusidate which is more stable than Fusidic acid
It has a more stable and quality enriched Fusidic acid as the final API
The Fusidic acid in the present invention degrades more slowly than the conventional products
The stability level of the Fusidic acid in the present invention remains within the acceptable limits throughout the shelf life of the product
The particle size of the Fusidic acid is finer and overall particle distribution in the cream is better, thereby providing better dermaceutical efficacy

While the above description contains much specificity, these should not be construed as limitation in the scope of the invention, but rather as an exemplification of the preferred embodiments thereof. It must be realized that modifications and variations are possible based on the disclosure given above without departing from the spirit and scope of the invention. Accordingly, the scope of the invention should be determined not by the embodiments illustrated, but by the appended claims and their legal equivalents.

1. A novel dermaceutical cream containing at least one corticosteroid, at least one antifungal, and Fusidic acid which is made in situ under oxygen-free environment using Sodium Fusidate, wherein said cream comprises Fusidic acid made in situ by a conversion of Sodium Fusidate, and a cream base containing at least one of each of a preservative, a primary and secondary emulsifier, a waxy material, a co-solvents, an acid, and water, preferably purified water.

2. A novel dermaceutical cream as claimed in claim 1, wherein said corticosteroid is added from about 0.001% (w/w) to about 5% (w/w), preferably from about 0.005% (w/w) to about 2.00% (w/w), and most preferably from about 0.05% (w/w) to 1.0% (w/w), and said antifungal is added from about 0.01% (w/w) to about 10% (w/w), preferably from about 0.1% (w/w) to about 5.00% (w/w), and most preferably from about 1% (w/w) to 2.0% (w/w) and said Fusidic acid is present in an amount from about 0.1% (w/w) to about 25% (w/w), preferably from about 0.5% (w/w) to about 5% (w/w), and more preferably about 2.00% (w/w), and in which the amount of said Sodium Fusidate used to form in situ said Fusidic acid is in the range between about 0.1% (w/w) to about 25% (w/w), preferably from about 0.5% (w/w) to about 5% (w/w) and more preferably about 2.08% (w/w), and
said preservative is selected from a group comprising Methylparaben, Propylparaben, Chlorocresol, Potassium sorbate, Benzoic acid and the like, either singly or any combination thereof, to form a proportion from about 0.05% (w/w) to 0.5% (w/w), preferably 0.3% (w/w), more preferably 0.2% (w/w),
said primary and secondary emulsifier is selected from a group comprising Cetostearyl alcohol, Cetomacrogol-1000, Polysorbate-80, Span-80 and the like, either singly or any combination thereof, to form a proportion from about 1% (w/w) to 15% (w/w), preferably 15% (w/w), more preferably 14.5% (w/w),
said waxy material is selected from a group comprising White soft paraffin, Liquid Paraffin, Hard paraffin and the like, either singly or any combination thereof, to form a proportion from about 5% (w/w) to 20% (w/w), preferably 15% (w/w), more preferably 12.5% (w/w),
said co-solvent is selected from a group comprising Propylene Glycol, Hexylene Glycol, PolyEthylene Glycol-400 and the like, either singly or any combination thereof, to form a proportion from about 5% (w/w) to 40% (w/w), preferably 30% (w/w), more preferably 25% (w/w),
said acid is selected from a group comprising acids such as HCl, H2So4, HNO3, Lactic acid and the like, either singly or any combination thereof, to form a proportion from about 0.005% (w/w) to 0.5% (w/w), preferably 0.3% (w/w), more preferably 0.25% (w/w), and
water in the amount in the range of 20% (w/w) to 75% (w/w), preferably 35% (w/w) to 50% (w/w), more preferably 38% (w/w) to 43% (w/w), preferably purified water.

3. A novel dermaceutical cream as claimed in claims 1 and 2 which further comprises a buffering agent, wherein said buffering agent is selected from a group comprising Di Sodium Hydrogen Ortho Phosphate, Sodium Hydrogen Ortho Phosphate and the like, either singly or any combination thereof, to form a proportion from about 0.01% (w/w) to 1.00% (w/w), preferably 0.5% (w/w), more preferably 0.05% (w/w).

4. A novel dermaceutical cream as claimed in claims 1 to 3 which further comprises an anti-oxidant, wherein said anti-oxidant is selected from a group comprising Butylated Hydroxy Anisole, Butylated Hydroxy Toluene and the like, either singly or any combination thereof, to form a proportion from about 0.001% (w/w) to 5% (w/w), preferably 0.1% (w/w), more preferably 0.01% (w/w).

5. A novel dermaceutical cream as claimed in claims 1 to 4 which further comprises a chelating agent, wherein said chelating agent is selected from a group comprising Disodium EDTA and the like, either singly or any combination thereof, to form a proportion from about 0.01% (w/w) to 1% (w/w), preferably 0.5% (w/w), more preferably 0.1% (w/w).

6. A novel dermaceutical cream as claimed in claims 1 to 5 which further comprises a humectant, wherein said humectant is selected from a group comprising Glycerin, Sorbitol, Propylene glycol and the like, either singly or any combination thereof, to form a proportion from about 5% (w/w) to 40% (w/w), preferably 30% (w/w), more preferably 25% (w/w).

7. A novel dermaceutical cream as claimed in claims 1 to 6 wherein sodium fusidate is converted in-situ under totally oxygen free environment by slow addition of an acid, into Fusidic acid of a molecular dispersion form (due to the presence of a co-solvent) at the intermediate stage, and which Fusidic acid regenerates into an extremely finely dispersed form when added to a final cream base, thereby resulting in a finely and homogeneously dispersed Fusidic acid in the final cream; all operations of converting sodium fusidate into Fusidic acid carried out preferably in an environment free of atmospheric oxygen.

8. A novel dermaceutical cream as claimed in claims 1 to 7 wherein said conversion of Sodium Fusidate into said Fusidic acid and the following formation of said Fusidic acid in a finely dispersed form in the final cream base take place in an oxygen-free environment.

9. A novel dermaceutical cream as claimed in claim 8 wherein said oxygen-free environment comprises a gaseous environment formed of inert gas selected from a group comprising carbon dioxide, nitrogen, helium and the like.

10. A method of treating primary & secondary bacterial skin infections, fungal skin infections and inflammations said method comprising applying of a cream containing Fusidic acid which is made in situ under oxygen-free environment using Sodium Fusidate, wherein said cream comprises Fusidic acid made using Sodium Fusidate, at least one corticosteroid, at least one antifungal, a cream base containing a preservative, primary and secondary emulsifiers, waxy materials, co-solvents, acids, and water.

11. A method of treating primary & secondary bacterial skin infections, fungal skin infections and inflammations said method comprising applying of a cream as described in claim 10, wherein said cream further comprises any of a group comprising a buffering agent, an anti oxidant, a chelating agent, and a humectant, or any combination thereof.

12. A method of treating primary & secondary bacterial skin infections, fungal skin infections and inflammations said method comprising applying of a cream as described in claim 2.

13. A method of treating primary & secondary bacterial skin infections, fungal skin infections and inflammations said method comprising applying of a cream as described in claim 3.

14. A method of treating primary & secondary bacterial skin infections, fungal skin infections and inflammations said method comprising applying of a cream as described in claim 4.

15. A method of treating primary & secondary bacterial skin infections, fungal skin infections and inflammations said method comprising applying of a cream as described in any of claims 5 to 7.
